(19) United States
(12) Patent Application Publication
(10) Pub. No.: US 2024/0409877 A1
(43) Pub. Date: Dec. 12, 2024

(54) MITOCHONDRIA ISOLATION FROM CELLS IN SUSPENSION

(71) Applicant: CELLERGY THERAPEUTICS LTD, Yavne (IL)

(72) Inventors: Ganit YARDEN, Gan Yavne (IL); Eyal KESNER, Efrat (IL); Keren KOHAVI BECK, Kfar Mordechai (IL)

(21) Appl. No.: 18/808,041

(22) Filed: Aug. 18, 2024

Related U.S. Application Data
(63) Continuation-in-part of application No. PCT/IL2024/050207, filed on Feb. 22, 2024.
(60) Provisional application No. 63/447,675, filed on Feb. 23, 2023.

(51) Int. Cl. C12N 1/06; A61K 35/12; A61P 1/16; A61P 3/10; C12N 5/0783

(52) U.S. Cl. CPC C12N 1/066; A61K 35/12; A61P 1/16; A61P 3/10; C12N 5/0636; C12N 2501/2302; C12N 2501/51; C12N 2501/515; C12N 2501/53

(57) ABSTRACT

Methods of producing a mitochondria extract comprising providing primary human hematopoietic cells in suspension, expanding the primary human hematopoietic cells, and isolating mitochondria are provided. Mitochondria extracts, compositions comprising mitochondria extract and methods of use of the extracts and compositions are also provided.

### CROSS REFERENCE TO RELATED APPLICATIONS

This application is a continuation-in-part of PCT Patent Application No. PCT/IL2024/050207 filed Feb. 22, 2024, which claims the benefit of priority of U.S. Provisional Patent Application No. 63/447,675, filed Feb. 23, 2023, the contents of which are all incorporated herein by reference in their entirety.

### FIELD OF INVENTION

The present invention is in the field of mitochondria isolation and transplantation.

### BACKGROUND OF THE INVENTION

Mitochondria play a key role in the homeostasis of the vast majority of the body's cells. Decreased mitochondrial function can cause a variety of diseases. In recent years, it has been reported that mitochondrial transplantation into cells, tissue, model systems and patients, either autologous or non-autologous, is not only possible but therapeutically effective. There has been limited research into the methods of mitochondria extraction that best retain mitochondrial function and structural integrity. There is currently an unmet need for effective and reproducible methods to produce/manufacture functional mitochondria in a therapeutic dose for treating diseases and disorders associated with nonfunctional or dysfunctional mitochondria.

### SUMMARY OF THE INVENTION

The present invention provides methods of producing a mitochondria extract comprising providing primary human hematopoietic cells in suspension, expanding the primary human hematopoietic cells, and isolating mitochondria. Mitochondria extracts, compositions comprising mitochondria extract and methods of use of the extracts and compositions are also provided.

According to a first aspect, there is provided a method for producing a mitochondria extract, the method comprising:
a. providing primary human hematopoietic cells in suspension;
b. expanding the primary human hematopoietic cells for a time sufficient to produce an expanded population of human hematopoietic cells comprising at least 10 times the number of primary human hematopoietic cells provided; and
c. isolating mitochondria from the expanded population; thereby producing a mitochondria extract.

According to another aspect, there is provided a method for producing a mitochondria extract, the method comprising:
a. providing a population of isolated primary human T cells in suspension;
b. activating the primary human T cells;
c. expanding the activated primary human T cells for a time sufficient to produce an expanded population of human T cells comprising at least 10 times the number of primary human T cells provided; and
d. isolating mitochondria from the expanded population;
thereby producing a mitochondria extract.

According to some embodiments, the primary hematopoietic cells are obtained from a blood sample from a human subject.

According to some embodiments, the primary hematopoietic cells are isolated from peripheral blood mononuclear cells (PBMCs).

According to some embodiments, the primary human hematopoietic cells are selected from T cells, B cells, NK cells and hematopoietic stem cells (HSCs).

According to some embodiments, the primary human hematopoietic cells are primary human immune cells.

According to some embodiments, the primary human immune cells are selected from T cells, B cells, and NK cells.

According to some embodiments, the primary human hematopoietic cells are an isolated population of T cells.

According to some embodiments, the population of isolated primary human T cells consists of at least 90% T cells.

According to some embodiments, the isolated primary T cells are isolated from peripheral blood mononuclear cells (PBMCs).

According to some embodiments, the time is between 7 and 14 days.

According to some embodiments, the expanded population of human hematopoietic cells comprises at least 20 times the number of primary human hematopoietic cells provided.

According to some embodiments, the expanded population of human hematopoietic cells comprises at least 50 times the number of primary human hematopoietic cells provided.

According to some embodiments, the expanded population of human T cells comprises at least 20 times the number of primary human T cells provided.

According to some embodiments, the expanded population of human T cells comprises at least 50 times the number of primary human T cells provided.

According to some embodiments, the isolated mitochondria comprise at least 20 micrograms of protein for every 1 million primary human hematopoietic cells provided.

According to some embodiments, the isolated mitochondria comprise at least 50 micrograms of protein for every 1 million primary human hematopoietic cells provided.

According to some embodiments, the isolated mitochondria comprise at least 80 micrograms of protein for every 1 million primary human hematopoietic cells provided.

According to some embodiments, the isolated mitochondria comprise at least 20 micrograms of protein for every 1 million primary human T cells provided.

According to some embodiments, the isolated mitochondria comprise at least 50 micrograms of protein for every 1 million primary human T cells provided.

According to some embodiments, the isolated mitochondria comprise at least 80 micrograms of protein for every 1 million primary human T cells provided.

According to some embodiments, the protein is determined by Bradford assay.

According to some embodiments, the activating comprises contacting the primary human T cells with an anti-CD3 antibody.

According to some embodiments, the expanding comprises contacting the provided primary human hematopoietic cells with at least one of: antigen presenting cells (APCs), components of APCs, components that mimic APC activity, a factor secreted by an activated T cell, and any combination thereof.

According to some embodiments, the provided primary human hematopoietic cells are primary T cells and wherein the expanding comprises contacting the primary T cells with at least one of an anti-CD3 antibody or antigen-binding fragment thereof, an anti-CD28 antibody or antigen-binding fragment thereof, an anti-CD2 antibody or antigen-binding fragment thereof, interleukin 2 (IL-2), and any combination thereof.

According to some embodiments, the expanding comprises contacting the primary T cells with an anti-CD3 antibody or antigen-binding fragment thereof, an anti-CD28 antibody or antigen-binding fragment thereof, an anti-CD2 antibody or antigen-binding fragment thereof, and IL-2.

According to some embodiments, the expanding comprises culturing the suspension of primary human hematopoietic cells in a gas-permeable vessel.

According to some embodiments, the culturing is in a bioreactor.

According to some embodiments, the gas-permeable vessel is a gas permeable rapid expansion (G-REX) well.

According to some embodiments, the isolating mitochondria comprises lysing cells to produce a cell lysate, wherein the lysing comprises at least one of addition of a lysis buff, needle shearing, homogenization with a Dounce homogenizer and nitrogen cavitation.

According to some embodiments, the isolating comprises disrupting a membrane of the immune cells by nitrogen cavitation or Dounce homogenization.

According to some embodiments, the isolating comprises disrupting a membrane of the immune cells by nitrogen cavitation.

According to some embodiments, the method comprises contacting the lysate with an artificial support comprising an anti-TOM22 binding agent and isolating the artificial support and any mitochondria bound thereto.

According to some embodiments, the method further comprises eluting the mitochondria from the artificial scaffold.

According to some embodiments, the method comprises centrifuging the lysate at about 3000 g to remove cellular debris and produce a supernatant and centrifuging the supernatant at about 12,000 g to produce a mitochondrial precipitate.

According to another aspect, there is provided a mitochondria extract produced by a method of the invention.

According to another aspect, there is provided a mitochondria extract which is at least 90% T cell mitochondria, at least 90% B cell mitochondria, at least 90% NK cell mitochondria or at least 90% HSC mitochondria.

According to another aspect, there is provided a mitochondria extract which is at least 90% human T cell mitochondria.

According to another aspect, there is provided a mitochondria extract which is at least 90% activated human T cell mitochondria.

According to some embodiments, the mitochondria extract is at least 95% T cell mitochondria.

According to another aspect, there is provided a pharmaceutical composition comprising a mitochondria extract of the invention.

According to some embodiments, the pharmaceutical composition further comprises a pharmaceutically acceptable carrier, excipient, or adjuvant.

According to some embodiments, the pharmaceutical composition is formulated for administration to a subject.

According to some embodiments, the pharmaceutical composition is formulated for in vitro transfer into a target cell.

According to another aspect, there is provided a recombinant cell comprising a mitochondria extract of the invention.

According to some embodiments, the recombinant cell is depleted of endogenous mitochondria.

According to some embodiments, the cell is a non-hematopoietic cell.

According to another aspect, there is provided a pharmaceutical composition comprising a recombinant cell of the invention and a pharmaceutically acceptable carrier, excipient or adjuvant.

According to another aspect there is provided a method of treating a subject suffering from a mitochondrial disease, the method comprising, administering to the subject a pharmaceutical composition of the invention, thereby treating the mitochondrial disease.

According to some embodiments, the mitochondrial disease is selected from diabetes, Parkinson disease, cancer, Alzheimer's disease, a genetic mitochondrial disorder, aging, and dilated cardiomyopathy.

According to some embodiments, the mitochondrial extract is autologous to the subject.

According to some embodiments, the mitochondrial extract is allogeneic to the subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: A line graph of T cell number and viability over 2 weeks of culture in a G-REX plate.

FIG. 2: A line graph of CS activity of isolated mitochondria from T cells from day 7, day 10 and day 13 of culture.

FIG. 3: Oxygen consumption rates (OCR) of isolated mitochondria with glutamate/malate as substrates, measured using a Seahorse Bioanalyzer.

FIGS. 4A-4B: (4A) Bar graph of membrane potential measurements of fresh isolated mitochondria, mitochondria after 4 days at 4° C. or at −80° C. (4B) Line graph of oxygen consumption rates of fresh isolated mitochondria, mitochondria after 4 days at 4° C. or at −80° C.

FIGS. 5A-5E: Micrographs showing H&E staining (Left) as well as staining with Oil Red O (Right, lipids are shown in red) for (5A) healthy control, group A, (5B) healthy control treated with mitochondria, group D, (5C) high fat diet untreated, group B, (5D) high fat diet treated with mitochondria, group E, (5E) high fat diet treated with a half regimen of mitochondria, group C.

FIGS. 6A-C: (6A) Seahorse assay on PBMCs, activated T cells, non-activated T cells and non-T cells from the PBMCs. (6B-C) Bar charts quantifying (6B) baseline oxygen consumption rate (OCR) and (6C) respiratory control ratio (RCR) based on the seahorse assay. RCR is the ratio of the oxygen consumption rate (OCR) in the presence of ADP (state 3 respiration) to the OCR in the absence of ADP (state 4 respiration). p-values were calculated by T-test. ****<0.0001; ***<0.001.

### DETAILED DESCRIPTION OF THE INVENTION

The present invention, in some embodiments, provides methods of producing a mitochondria extract comprising providing primary human hematopoietic cells in suspension, expanding the primary human hematopoietic cells, and isolating mitochondria. The present invention further concerns mitochondria extracts, compositions comprising mitochondria extracts and method of using the extracts and compositions.

According to one aspect, the present invention discloses a method for providing functional mitochondria from non-transformed primary hematopoietic cells in suspension (e.g., immune cells, activated T cells). Advantageously, the amount of hematopoietic cells-derived functional mitochondria obtained from a single blood unit, based on the method of the invention, is sufficient for at least one therapeutic dose, and also for multiple therapeutic doses. Multiple therapeutic doses may be provided for repeat dosing for the same patient or for several different patients. In some embodiments, the method produces a sufficient number of functional mitochondria to produce multiple doses of a therapeutic composition of mitochondria. In some embodiments, the composition is a composition of the invention. In some embodiments, multiple doses is at least 1, 2, 3, 4, 5, 10, 15 20, 30, 40, 45, 50, 55, 60, 70, 80, 90, 100, 110, 120, 130, 140, or 150 therapeutic doses. Each possibility represents a separate embodiment. In some embodiments, the multiple doses are generated from a single blood unit. In some embodiments, a single blood unit is a single blood sample. In some embodiments, a single blood sample is a single blood draw. In some embodiments, the generating is by a method of the invention. In some embodiments, multiple doses is at least 2 doses. In some embodiments, multiple doses is at least 5 doses. In some embodiments, multiple doses is at least 10 doses. In some embodiments, multiple doses is at least 100 doses. It will be understood by a skilled artisan, that as the cells themselves are not intended to be used for cell therapy, their functionality, therapeutic potential, and/or cytotoxic activity in the culture is not a limiting factor, rather merely the quality of the mitochondria. Thus, the cells from a single blood unit can be grown as long as the mitochondria quality and/or function is maintained. In this way the method of the invention has the enormous advantage of producing very large amounts of mitochondria.

The invention is based at least in part on the surprising yield of mitochondria that was produced from primary activated T cells in culture. The method of the invention allowed for the production of more than 85 ug of protein of isolated mitochondria from a starting population of just 1 million primary T cells. Further, optimizations of the method produced yields even as high as 220 ug protein per 1 million primary T cells. When the optimized method is performed in a bioreactor and not just tissue culture plates, yields up to and exceeding 1000 ug are reached. The use of hematopoietic cells in suspension for the generation of mitochondria allowed for a cellular expansion not possible with primary culture of adherent cells. Further, these mitochondria were of high quality, fully functional and suitable for therapeutic use in a human subject. The produced composition was able to effectively treat diabetic and fatty liver mice.

By a first aspect, there is provided a method for producing a mitochondria extract, the method comprising:
a. providing cells in suspension;
b. expanding the cells for a time sufficient to produce an expanded population of cells; and
c. isolating mitochondria from the expanded population;
   thereby producing a mitochondria extract.

As used herein, the term “mitochondria extract” refers to a composition of extracellular mitochondria. Extracellular mitochondria refer to mitochondria that are outside of a cell, that is, that are not enclosed by a plasma/cellular membrane. These mitochondria have been extracted from cells and are thus a mitochondria extract. In some embodiments, the mitochondria are intact. In some embodiments, intact is structurally intact. In some embodiments, the mitochondria are functional. In some embodiments, the mitochondria extract is functional. As used herein, the term “functional” with respect to mitochondria refers to the function of oxidative phosphorylation and/or ATP synthesis. In some embodiments, the mitochondria are capable of oxidative phosphorylation. In some embodiments, the mitochondria are healthy. In some embodiments, the mitochondria are as functional as control mitochondria. In some embodiments, control mitochondria are mitochondria from healthy cells. In some embodiments, healthy cells are cells in suspension. In some embodiments, healthy cells are healthy immune cells. In some embodiments, healthy cells are healthy T cells. In some embodiments, the functional mitochondria perform oxidative phosphorylation at a rate at or above a predetermined threshold. In some embodiments, the predetermined threshold is the rate of oxidative phosphorylation of mitochondria from control cells. In some embodiments, the predetermined threshold is the rate of oxidative phosphorylation in mitochondria isolated from control cells.

Methods and kits for measuring oxidative phosphorylation are provided hereinbelow and also are well known in the art. Assays that can be used include assays measuring oxygen consumption rate, ATP production assays, mitochondrial membrane potential measurements (dyes such as JC-1, TMRM and rhodamine 123 may be used), enzyme activity assays (for measuring activity of the electron transport chain), high resolution respirometry and NADH/NAD+ ratio measurement. In some embodiments, oxidative phosphorylation is as measured by the seahorse assay.

In some embodiments, the rate of oxidative phosphorylating is the oxygen consumption rate (OCR). In some embodiments, the rate of oxidative phosphorylating is the basal respiration rate. In some embodiments, the rate of oxidative phosphorylating is the basal OCR. In some embodiments, functional mitochondria comprise a basal OCR of at least a predetermined threshold. Healthy and functional mitochondria have a measurable basal respiration, while very low or zero basal OCR is indicative of damaged or non-functional mitochondria. In some embodiments, the rate of oxidative phosphorylation is the rate of ADP-stimulated respiration. ADP-stimulated respiration refers to the OCR after ADP is added to the mitochondria. In some embodiments, functional mitochondria comprise an ADP-stimulated respiration of at least a predetermined threshold. In some embodiments, functional mitochondria comprise an increase from basal OCR to ADP-stimulated OCR of at least a predetermined threshold. A significant increase in OCR after ADP addition is indicative of functional ATP synthesis and electron transport chain. In some embodiments, functional mitochondria comprise a State 4 respiration rate below at or below a predetermined threshold. State 4 respiration is OCR measured after all ADP is converted to ATP. High State 4 respiration is indicative of mitochondrial uncoupling or damage. In some embodiments, the rate of oxidative phosphorylation is the maximal respiratory capacity. Maximum capacity refers to OCR after addition of an uncoupler (e.g., FCCP). In some embodiments, functional mitochondria comprise a maximal respiratory capacity of at least a predetermined threshold. A high maximal respiration indicates mitochondria capable of high electron transport activity. In some embodiments, the rate of oxidative phosphorylation is the spare respiratory capacity. In some embodiments, functional mitochondria comprise a spare respiratory capacity of at least a predetermined threshold. Spare capacity refers to the difference between the maximal respiration and the basal respiration. A low spare capacity indicates the mitochondria are compromised. In some embodiments, functional mitochondria comprise a coupling efficiency of at least a predetermined threshold. Coupling efficiency is the percentage of basal respiration used for ATP production. High coupling indicates healthy mitochondria while low efficiency indicates uncoupling and damage. In some embodiments, the rate of oxidative phosphorylation is the respiratory control rate (RCR). RCR is the ratio of the State 3 (ADP-stimulated) OCR to the State 4 OCR. In some embodiments, functional mitochondria comprise an RCR of at least a predetermined threshold. A high RCR indicates tightly coupled mitochondria with efficient ATP production. In some embodiments, the predetermined threshold is an RCR of 5. In some embodiments, functional mitochondria comprise an RCR of at least 5. In some embodiments, functional mitochondria comprise an RCR of greater than 5. RCR is the most common measure used for evaluating functionality of isolated mitochondria. In some embodiments, the predetermined threshold is the rate in control mitochondria. In some embodiments, the predetermined threshold is the rate in mitochondria from control cells. In some embodiments, control mitochondria are healthy mitochondria.

In some embodiments, the method further comprises the step of preserving the produced mitochondria extract. In some embodiments, preserving techniques are selected from: cryopreservation, thawing, freezing, vitrification, dry state preservation, hypothermic storage, normothermic storage, encapsulation, entrapment in a matrix, and encapsulation and entrapment in a polymer. In some embodiments, preserving is freezing. In some embodiments, preserving is entrapping. In some embodiments, preserving is encapsulating. In some embodiments, preserving is drying. In some embodiments, preserving is freeze drying.

In some embodiments, the method further comprises the step of providing the mitochondria extract or the preserved mitochondria extract to a subject in need thereof. In some embodiments, providing is administering.

In some embodiments, the method is an in vitro method. In some embodiments, the method is an ex vivo method. In some embodiments, the method is a method of culture. In some embodiments, the method is a method of cell culture. In some embodiments, the culture is in suspension. In some embodiments, the culture is in a tissue culture plate or well. In some embodiments, the culture is in a bioreactor.

In some embodiments, a mitochondria extract comprises isolated mitochondria. In some embodiments, isolated is isolated from a cell. In some embodiments, the cell is a healthy cell. In some embodiments, the cell is a suspension cell. In some embodiments, the cell is an immune cell. In some embodiments, a mitochondria extract is isolated mitochondria. In some embodiments, isolated mitochondria are purified mitochondria. In some embodiments, the extract is at least 70, 75, 80, 85, 86, 87, 88, 89, 90, 91, 92, 93, 94, 95, 96, 97, 98, 99 or 100% mitochondria. Each possibility represents a separate embodiment of the invention. In some embodiments, the isolated mitochondria comprise functional mitochondria. In some embodiments, the isolated mitochondria are functional mitochondria. In some embodiments, a mitochondria extract is isolated mitochondria comprising functional mitochondria. In some embodiments, a mitochondria extract is isolated mitochondria comprising intact mitochondria. In some embodiments, a mitochondria extract comprises functional mitochondria. In some embodiments, a mitochondria extract comprises intact functional mitochondria. In some embodiments, the extract is at least 70, 75, 80, 85, 86, 87, 88, 89, 90, 91, 92, 93, 94, 95, 96, 97, 98, 99 or 100% functional mitochondria. Each possibility represents a separate embodiment of the invention. In some embodiments, the extract is at least 70, 75, 80, 85, 86, 87, 88, 89, 90, 91, 92, 93, 94, 95, 96, 97, 98, 99 or 100% intact mitochondria. Each possibility represents a separate embodiment of the invention. In some embodiments, the extract is at least 90% mitochondria. In some embodiments, the extract comprises a purity of at least 70, 75, 80, 85, 90, 95, 97, 99 or 100%. Each possibility represents a separate embodiment of the invention. In some embodiments, the extract comprises a purity of at least 90%.

In some embodiments, the cells are mammalian cells. In some embodiments, the cells are human cells. In some embodiments, the cells are primary cells. In some embodiments, the cells are obtained from a subject. In some embodiments, providing is obtaining. In some embodiments, obtaining is providing. In some embodiments, the cells are directly obtained from a subject. In some embodiments, the cells are differentiated from stem cells. In some embodiments, the stem cells are embryonic stem cells. In some embodiments, the embryonic stem cells are cord blood derived stem cells. In some embodiments, the stem cells are induced pluripotent stem cells (iPSCs). In some embodiments, the stem cells are hematopoietic stem cells (HSCs). In some embodiments, the method further comprises extracting the primary cells from a subject. In some embodiments, the subject is human. In some embodiments, the cells are from a healthy subject. In some embodiments, the subject does not suffer from a mitochondrial disease. In some embodiments, the subject suffers from a condition that can benefit from increased mitochondrial function.

In some embodiments, the cells are primary cells. In some embodiments, the cells are not immortalized. In some embodiments, the cells are not cells of a cell line. In some embodiments, the cells are not transformed. In some embodiments, the cells are healthy cells. In some embodiments, the cells are not cancerous. The term “primary cells” is well known in the art and refers to cells taken directly from a living organism. In some embodiments, the cells are from a sample provided by a subject. In some embodiments, the method further comprises receiving a sample from a subject. In some embodiments, the method further comprises taking a sample from a subject. In some embodiments, the sample is a sample comprising cells. In some embodiments, the sample comprises a bodily fluid. In some embodiments, the bodily fluid is selected from: blood, serum, plasma, gastric fluid, intestinal fluid, saliva, bile, breast milk, urine, interstitial fluid, cerebral spinal fluid and stool. In some embodiments, the fluid is blood. In some embodiments, the blood is peripheral blood. In some embodiments, the primary cells are obtained from a blood sample. In some embodiments, the method also comprises differentiating the obtained cells into the hematopoietic cells in suspension. In some embodiments, the method also comprises differentiating iPSCs into hematopoietic cells.

In some embodiments, the cells are suspension cells. In some embodiments, the cells are non-adherent cells. In some embodiments, the cells are grown in suspension. In some embodiments, the cells are hematopoietic cells. In some embodiments, the hematopoietic cells are blood cells. In some embodiments, the hematopoietic cells are isolated from peripheral blood mononuclear cells (PBMCs). In some embodiments, the hematopoietic cells are differentiated from stem cells. In some embodiments, the hematopoietic cells are selected from T cells, B cells, natural killer (NK) cells and hematopoietic stem cells (HSCs). In some embodiments, the cells are immune cells. In some embodiments, the hematopoietic cells are immune cells. In some embodiments, the immune cells are lymphoid cells. In some embodiments, the cells are lymphocytes. In some embodiments, the immune cells are selected from T cells, B cells, and NK cells. In some embodiments, the cells are T cells. In some embodiments, the cells are B cells. In some embodiments, the cells are NK cells. In some embodiments, the cells are HSCs. In some embodiments, the cells are human cells. In some embodiments, the cells are from a subject. In some embodiments, the cells are activated cells. In some embodiments, the cells are activated T cells.

In some embodiments, the cells are a mix of hematopoietic cells. In some embodiments, the cells are an isolated population of cells. In some embodiments, the cells are a homogenous population of cells. In some embodiments, the cells are an enriched population of cells. In some embodiments, isolated is purified. In some embodiments, isolated is enriched. In some embodiments, an enriched population comprises at least 20, 30, 40, 50, 60, 70, 75, 80, 85, 90, 95, 97, 99 or 100% homogenous cell type. Each possibility represents a separate embodiment of the invention. In some embodiments, a purified population comprises at least 20, 30, 40, 50, 60, 70, 75, 80, 85, 90, 95, 97, 99 or 100% purity. Each possibility represents a separate embodiment of the invention. In some embodiments, an enriched or purified population comprises at least 90% purity. In some embodiments, the cells are an isolated population of T cells. In some embodiments, the cells are an isolated population of B cells. In some embodiments, the cells are an isolated population of NK cells. In some embodiments, the cells are an isolated population of HSCs. In some embodiments, the cells are an enriched population of T cells. In some embodiments, the cells are an enriched population of B cells. In some embodiments, the cells are an enriched population of NK cells. In some embodiments, the cells are an enriched population of HSCs. In some embodiments, the cells are a mix of hematopoietic cells comprising at least 2, 3, 4 or 5 different types of cells. Each possibility represents a separate embodiment of the present invention. In some embodiments, the cells are a mix of B cells and additional hematopoietic cells. In some embodiments, the cells are a mix of B cells and additional PBMCs. In some embodiments, the cells are a mix of T cells and additional hematopoietic cells. In some embodiments, the cells are a mix of T cells and additional PBMCs. In some embodiments, the cells are a mix of NK cells and additional hematopoietic cells. In some embodiments, the cells are a mix of NK cells and additional PBMCs. In some embodiments, the cells are a mix of immune cells comprising at least 2, 3, 4, 5 types of different immune cells. Each possibility represents a separate embodiment of the present invention.

In some embodiments, the method comprises determining the cell types of the obtained population prior to expansion. In some embodiments, the method comprises isolating a specific cell type from the obtained population prior to expansion.

In some embodiments, the cells are expanded in solution. In some embodiments, the cells are expanded in culture. In some embodiments, expanding is culturing. In some embodiments, the cells are expanded for at least a predetermined time. In some embodiments, the cells are expanded for a time sufficient to produce an expanded population. In some embodiments, the time is at least 4, 5, 6, 7, 8, 9, or 10 days. Each possibility represents a separate embodiment of the invention. In some embodiments, the time is at least 7 days. In some embodiments, the time is at most 10, 11, 12, 13, 14, 15, 16, 17 or 18 days. In some embodiments, the time is at most 10 days. In some embodiments, the time is at most 14 days. In some embodiments, the time is at most 13 days. In some embodiments, the time is between 7-14 days. In some embodiments, the time is between 7-13 days. In some embodiments, the time is between 7-10 days. In some embodiments, the time is between 10-14 days. In some embodiments, the time is between 10 and 13 days.

In some embodiments, the expanded population comprise at least 2, 3, 4, 5, 6, 7, 8, 9, 10, 11, 12, 13, 14, 15, 20, 25, 30, 35, 40, 45, 50, 60, 70, 80, 90, 100, 150, 200,250, 300, 400, 500, or 1000 times the number of cells provided. Each possibility represents a separate embodiment of the invention. In some embodiments, the expanded population comprises at least 5 times the number of cells provided. In some embodiments, the expanded population comprises at least 10 times the number of cells provided. In some embodiments, the expanded population comprises at least 20 times the number of cells provided. In some embodiments, the expanded population comprises at least 30 times the number of cells provided. In some embodiments, the expanded population comprises at least 100 times the number of cells provided. In some embodiments, the expanded population comprises at least 200 times the number of cells provided. In some embodiments, the expanded population comprises at least 400 times the number of cells provided. In some embodiments, the expanded population comprises at least 1000 times the number of cells provided. In some embodiments, the time is sufficient to produce at least a 2-, 3-, 4-, 5-, 6-, 7-, 8-, 9-, 10-, 11-, 12-, 13-, 14-, 15-, 20-, 25-, 30-, 35-, 40-, 45-, 50-, 100-, 200-, 300-, 400-, 500-, 600-, 700-, 800-, 900-, or 1000-fold expansion of the cells provided. Each possibility represents a separate embodiment of the invention. In some embodiments, the time is sufficient to produce at least a 2-fold expansion of the cells provided. In some embodiments, the time is sufficient to produce at least a 3-fold expansion of the cells provided. In some embodiments, the time is sufficient to produce at least a 4-fold expansion of the cells provided. In some embodiments, the time is sufficient to produce at least a 5-fold expansion of the cells provided. In some embodiments, the time is sufficient to produce at least a 100-fold expansion of the cells provided. In some embodiments, the time is sufficient to produce at least a 200-fold expansion of the cells provided. In some embodiments, the time is sufficient to produce at least a 300-fold expansion of the cells provided. In some embodiments, the time is sufficient to produce at least a 400-fold expansion of the cells provided. Methods of cell counting and determining cell number are well known in the art and it is routine for a skilled artisan to count the number of cells initially plated (the provided cells) and the number of cells present after the expansion (the expanded population).

In some embodiments, the cells are cultured in media. In some embodiments, the media is tissue culture media. In some embodiments, the media is suspension cell media. In some embodiments, the media is immune cell media. In some embodiments, the media is chemically defined media. In some embodiments, the media is T cell media. Suspension cell media in general and T cell media in specific are well known in the art and any such media may be used. Examples of commercially available medias for use in a method of the invention include RMPI basal media supplemented with 10% fetal bovine serum (FBS), CST™ OpTmizer™ T-cell media (ThermoFisher), TexMACS™ media (Miltenyi Biotec), X-VIVO 15 (Lonza), and 4Cell NutriT media (Gibco). In some embodiments, the media is 4Cell NutriT media. In some embodiments, the media is animal component-free media. In some embodiments, the media is a chemically defined media.

In some embodiments, the expanding comprises activating the cells. In some embodiments, the expanding comprises activating the immune cells. Methods of immune cell activation are well known in the art and any such methods, reagents or kits known for the purpose of activation may be employed. In some embodiments, the isolated T cells are activated. In some embodiments, T cells within a population of PBMCs are activated. In some embodiments, the activation is a T cell specific activation. Activations for T cells that do not activate other immune cells are well known and any such activation may be used. Well known methods of activating include, but are not limited to contact with anti-CD3, anti-CD28, anti-CD2, anti-CD137 (4-1BB), anti-CD27, anti-OX40 (CD134), anti-ICOS (CD278), anti-CD45RO, and anti-CD16 (FcgammaRIII). Combinations of these activating agents are also well known and may be used, such as for example anti-CD3 and anti-CD28 or anti-CD3, anti-CD28 and anti-CD2. In some embodiments, the activating comprises stimulating the CD3 receptor on the T cells. In some embodiments, the activating is by CD3. In some embodiments, the activating comprises stimulating the CD28 receptor on the T cells. In some embodiments, the activating is by CD28. In some embodiments, the activating comprises stimulating the CD2 receptor on the T cells. In some embodiments, the activating is by CD2. Anti-CD3, anti-CD28 and anti-CD2 antibodies and beads comprising the antibodies which can be used to activate T cells are well known in the art. This class of antibodies and their use in activating T cells are well known and a skilled artisan would be readily able to identify such antibodies as can be used.

In some embodiments, the expanding comprises contacting the provided cells with antigen presenting cells (APCs). In some embodiments, the expanding comprises contacting the provided cells with components of APCs. In some embodiments, the expanding comprises contacting the provided cells with components that mimic APC activity. In some embodiments, the component is an anti-CD2 antibody or antigen binding factor thereof. In some embodiments, activated is activated by anti-CD2. In some embodiments, the component is an anti-CD3 antibody or antigen binding factor thereof. In some embodiments, activated is activated by anti-CD3. In some embodiments, the component is an anti-CD28 antibody or antigen binding factor thereof. In some embodiments, activated is activated by anti-CD28. In some embodiments, the APC mimic is a bead or particle comprising a component of an APC. In some embodiments, the bead is an avidin bead. In some embodiments, the avidin is streptavidin. In some embodiments, an antibody or antigen binding factor thereof is a biotinylated antibody or antigen binding factor thereof. In some embodiments, the avidin bead has conjugated to its surface a biotinylated antibody or antigen binding factor thereof selected from anti-CD2, anti-CD3, anti-CD28 and a combination thereof. In some embodiments, the expanding comprises contacting the provided cells with a factor secreted by activated T cells. In some embodiments, the factor is Interleukin 2 (IL-2). In some embodiments, the expanding comprises contacting the provided cells with IL-2. In some embodiments, activated is activated by IL-2. In some embodiments, the IL-2 is present at a concentration of between 10-600 IU/mL. In some embodiments, the IL-2 is present at a concentration of about 200 IU/mL. In some embodiments, the factor is IL-7. In some embodiments, the factor is IL-15. In some embodiments, the factor is IL-7 and IL-15. In some embodiments, the expanding comprises contacting the provided cells with IL-7. In some embodiments, the expanding comprises contacting the provided cells with IL-15. In some embodiments, the expanding comprises contacting the provided cells with IL-7 and IL-15. In some embodiments, the expanding comprises contacting the provided cells with an anti-CD3 antibody or antigen binding fragment thereof. In some embodiments, the antibody is an activating antibody. In some embodiments, the expanding comprises contacting the provided cells with an anti-CD28 antibody or antigen binding fragment thereof. In some embodiments, the expanding comprises contacting the provided cells with an anti-CD2 antibody or antigen binding fragment thereof. In some embodiments, the expanding comprises contacting the cells with a combination of an anti-CD3 antibody, an anti-CD2 antibody, an anti-CD28 antibody and IL-2. In some embodiments, the expanding comprises contacting the cells with an anti-CD3 antibody, an anti-CD2 antibody, an anti-CD28 antibody and IL-2. In some embodiments, the factor is replaced in the culture every 1, 2, 3, 4, 5, 6, or 7 days. Each possibility represents a separate embodiment of the invention. In some embodiments, the factor is replaced in the culture every 3 days. Methods of expanding T cells are well known in the art and any such method may be used. Examples of agents that can used to expand T cells include, but are not limited to IL-2, IL-7, IL-15, IL-21, IL-4, IL-10, and transforming growth factor-beta (TGFB). Various combinations of the expanding agents are also known, and include, for example IL-2 and IL-7/IL-15, IL-15 and IL-21, and IL-7 and IL-15.

In some embodiments, the expanding comprises culturing. In some embodiments, the culturing is in suspension. In some embodiments, the method comprises culturing the suspension of cells in a gas permeable vessel. In some embodiments, the culturing is in a gas permeable vessel. In some embodiments, the culturing is in a culture bag. In some embodiments, the culturing is in a gas permeable bag. In some embodiments, the vessel is a well. In some embodiments, the vessel is a plate. In some embodiments, the vessel is a flask. In some embodiments, the vessel is sealed, but gas permeable. In some embodiments, at least one wall of the vessel is gas permeable. In some embodiments, a gas permeable vessel allows for unlimited exchange of gases to the suspension media. In some embodiments, the gas permeable vessel allows for unlimited oxygen to the cells. In some embodiments, the gas permeable vessel is a gas permeable rapid expansion (G-REX) vessel. In some embodiments, the G-REX vessel is a G-REX well. In some embodiments, the method comprises culturing the suspension of cells in a bioreactor. Examples of bioreactors include, but are not limited to Stirred Tank Bioreactor, Wave-Mixed Bioreactor, Fixed-Bed Bioreactor, Microcarrier-Based Bioreactor, Hollow Fiber Bioreactor, G-Rex Bioreactor and Perfusion Bioreactor System.

In some embodiments, the expanded population is a mix of hematopoietic cells. In some embodiments, the expanded population is a pure population. In some embodiments, the expanded population is an enriched population. In some embodiments, a pure population comprises at least 30, 40, 50, 60, 70, 75, 80, 85, 90, 95, 97, 99 or 100% purity. Each possibility represents a separate embodiment of the invention. In some embodiments, an enriched population comprises at least 30, 40, 50, 60, 70, 75, 80, 85, 90, 95, 97, 99 or 100% homogeneity in the population. Each possibility represents a separate embodiment of the invention. In some embodiments, a pure population comprises at least 90% purity. In some embodiments, an enriched population comprises at least 90% homogeneity. In some embodiments, the purity and/or homogeneity is with respect to the cell types present in the population. In some embodiments, the expanded population is an essentially pure population of cells of a specific cell type. In some embodiments, the expanded population is an essentially homogenous population of cells of a specific cell type. In some embodiments, the expanded population is an expanded population of T cells. In some embodiments, the expanded population is an expanded population of B cells. In some embodiments, the expanded population is an expanded population of NK cells. In some embodiments, the expanded population is an expanded population of HSCs. In some embodiments, the method further comprises measuring the purity of the population after the expanding. In some embodiments, the method comprises isolating a specific cell type from the expanded population. In some embodiments, after measuring only a pure population is used for mitochondria isolation.

In some embodiments, isolating is isolating a cell type. In some embodiments, isolating is isolating mitochondria. In some embodiments, isolating is purifying. In some embodiments, isolating is extracting. In some embodiments, isolating is isolating from cells. In some embodiments, isolating comprises disrupting a membrane of the cells. In some embodiments, isolating comprises disrupting a membrane of the cells without substantially disrupting a membrane of the mitochondria. In some embodiments, isolating comprises lysing the cells. In some embodiments, lysing the cells produces a lysate. In some embodiments, the lysate is a cellular lysate. In some embodiments, disrupting a membrane of a cell is lysing the cell. Methods of mitochondrial isolation from cells are well known and any method that removes intact and functional mitochondria from the cells may be used. For example, a commercially available mitochondrial isolation kit is available from Miltenyi (130-094-532), though any commercially available kit may be used as part of a method of the invention. In some embodiments, lysing is without substantially lysing mitochondria. In some embodiments, membrane disruption is plasma membrane disruption. In some embodiments, plasma membrane disruption does not substantially disrupt mitochondrial membranes. In some embodiments, the disrupting a plasma membrane is by nitrogen cavitation. In some embodiments, the disrupting a plasma membrane is by homogenization. In some embodiments, the homogenization is with a homogenizer. In some embodiments, the homogenizer is a Dounce homogenizer. In some embodiments, the homogenization is Dounce homogenization. In some embodiments, disrupting a membrane is by addition of a lysis buffer. In some embodiments, the lysis buffer comprises a detergent. In some embodiments, the lysis buffer lysis by osmotic pressure. In some embodiments, disrupting a membrane is by needle shearing. In some embodiments, the needle is about a 30-gage needle.

In some embodiments, the isolation comprises centrifuging the cells to remove media. In some embodiments, the cells are washed. In some embodiments, the washing is in wash buffer. In some embodiments, the wash buffer is PBS. In some embodiments, the centrifuging is at about 300 g. In some embodiments, the centrifuging is for a time sufficient to pellet the cells. In some embodiments, a sufficient time is about 1, 2, 3, 4, 5, 6, 7, 8, 9, 10, 12, 15, 17, or 20 minutes. Each possibility represents a separate embodiment of the invention. In some embodiments, a sufficient time is between 5-15 minutes. In some embodiments, a sufficient time is between 5-10 minutes. In some embodiments, a sufficient time is about 10 minutes. In some embodiments, cells are suspended in mitochondrial isolation buffer before lysing/membrane disruption. Options for isolation buffers include, but are not limited to sugars, HEPES, Tris-HCl, EDTA, EGTA, Magnesium Chloride (MgCl2), Potassium Chloride (KCl), Phosphate, Glutamate, Malate, ATP, Dithiothreitol (DTT) and Reduced Glutathione (GSH) buffers. In some embodiments, the isolation buffer is a sugar buffer. In some embodiments, the sugar is a monosaccharide. In some embodiments, the sugar is a disaccharide. In some embodiments, the sugar is a polysaccharide. In some embodiments, the isolation buffer is a sucrose buffer. In some embodiments, the isolation buffer comprises about 320 mM sucrose, about 5 mM Tris-HCl, and about 2 mM EGTA. In some embodiments, the isolation buffer is a mannitol buffer. In some embodiments, the isolation buffer is a trehalose buffer. In some embodiments, the isolation buffer comprises a neutral pH. In some embodiments, the isolation buffer comprises a very lowly basic pH. In some embodiments, the isolation buffer comprises a pH of about 7.4. In some embodiments, the isolation buffer comprises fatty acid-free bovine serum albumen (BSA). In some embodiments, the BSA concentration is about 0.5%. In some embodiments, mitochondria isolation comprises centrifuging the cell lysate. In some embodiments, the centrifuging is to remove cellular debris.

In some embodiments, the method comprises contacting the lysate with an anti-mitochondrial protein antibody or antigen binding fragment thereof. In some embodiments, contacting comprises incubating. In some embodiments, incubating is for a time sufficient to bind the mitochondrial protein to the antibody or antigen binding fragment thereof. In some embodiments, the mitochondrial protein is TOM22. In some embodiments, antibody or antigen binding fragment thereof is conjugated to a solid support. In some embodiments, the solid support is an artificial solid support. In some embodiments, the antibody or antigen binding fragment thereof is immobilized to a solid support. In some embodiment, the solid support is a synthetic solid support. In some embodiments, the solid support is a non-natural solid support. In some embodiments, the solid support is a man-made solid support. In some embodiments, the solid support is a column. In some embodiments, the solid support is a bead. In some embodiments, the bead is a magnetic bead. In some embodiments, the bead is a paramagnetic bead. In some embodiments, the bead is configured for isolation. In some embodiments, the bead is isolatable on a column. In some embodiments, the bead is an avidin bead. In some embodiments, avidin is streptavidin. In some embodiments, the isolating comprises isolating the solid support and any mitochondria bound thereto. In some embodiments, the isolating further comprises eluting the mitochondria from the solid support. In some embodiments, the eluting is with an elution buffer. In some embodiments, the elution buffer is a salt buffer. In some embodiments, a salt buffer is a high salt buffer. In some embodiments, the salt concentration is sufficiently high to elute the mitochondria from the antibody or antigen binding fragment thereof.

In some embodiments, the isolating comprises centrifuging the lysate. In some embodiments, the centrifuging is at a speed sufficient to precipitate cellular debris from the lysate. In some embodiments, the centrifuging is for a time sufficient to precipitate cellular debris from the lysate. In some embodiments, the sufficient speed is about 3000 g. In some embodiments, the sufficient time is about 5 minutes. In some embodiments, the centrifugation to remove the cellular debris is repeated. In some embodiments, the centrifuging produces a supernatant. In some embodiments, the supernatant comprises mitochondria. In some embodiments, the lysate is substantially depleted of cellular debris. In some embodiments, the lysate is substantially depleted of organelles other than mitochondria. In some embodiments, the isolating further comprises centrifuging the supernatant. In some embodiments, the centrifuging is at a speed sufficient to precipitate the mitochondria. In some embodiments, the centrifuging is for time sufficient to precipitate the mitochondria. In some embodiments, a sufficient speed is about 12,000 g. In some embodiments, a sufficient time is about 10 minutes. In some embodiments, precipitating mitochondria comprises producing a mitochondrial precipitate. In some embodiments, precipitating the mitochondria comprises producing a mitochondrial pellet. In some embodiments, a precipitate is a pellet. In some embodiments, the isolating further comprises resuspending the mitochondrial pellet in isolation buffer.

In some embodiments, the isolated mitochondria are a mitochondria extract. In some embodiments, the isolated mitochondria are intact mitochondria. In some embodiments, the isolated mitochondria are functional mitochondria. In some embodiments, at least 30, 40, 50, 55, 60, 65, 70, 75, 80, 85, 90, 92, 95, 97, 99 or 100% of the mitochondria are functional. Each possibility represents a separate embodiment of the invention. In some embodiments, function comprises ATP production. In some embodiments, function comprises oxygen consumption. In some embodiments, function comprises membrane potential. In some embodiments, function comprises citrate synthase. Methods of testing the functionality of mitochondria, including testing the functions listed hereinabove, are well known in the art and are also described hereinbelow. A skilled artisan is thus fully capable of determining the functional capabilities of the extracted and/or isolated mitochondria. In some embodiments, the method further comprises testing the quality of the mitochondria extract. In some embodiments, the cell lysate is tested. In some embodiments, the isolated mitochondria are tested. In some embodiments, the intact cells are tested. In some embodiments, the testing determines the quality of the mitochondria extract. In some embodiments, the testing comprises a citrate synthase assay. In some embodiments, the testing comprises a JC-1 assay. In some embodiments, the testing comprises determining mitochondrial DNA (mtDNA) copy number. In some embodiments, quantifying mitochondria in the extract is by mtDNA copy number. In some embodiments, quantifying the mitochondria in the extract is by the total amount of protein present after mitochondrial isolation/extraction.

In some embodiments, the isolated mitochondria comprise at least 10, 15, 20, 25, 30, 35, 40, 45, 50, 55, 60, 65, 70, 75, 80, 85, 90, 95, 100, 105, 110, 115, 120, 125, 130, 140, 145, 150, 155, 160, 165, 170, 175, 180, 185, 190, 195, 200, 220, 250, 300, 350, 400, 450, 500, 550, 600, 650, 700, 750, 800, 850, 900, 950, or 1000 micrograms (ug) of protein for every 1 million provided cells. Each possibility represents a separate embodiment of the invention. In some embodiments, the isolated mitochondria comprise at least 20 ug of protein for every 1 million cells provided. In some embodiments, the isolated mitochondria comprise at least 50 ug of protein for every 1 million cells provided. In some embodiments, the isolated mitochondria comprise at least 80 ug of protein for every 1 million cells provided. In some embodiments, the isolated mitochondria comprise at least 100 ug of protein for every 1 million cells provided. In some embodiments, the isolated mitochondria comprise at least 110 ug of protein for every 1 million cells provided. In some embodiments, the isolated mitochondria comprise at least 200 ug of protein for every 1 million cells provided. In some embodiments, the isolated mitochondria comprise at least 220 ug of protein for every 1 million cells provided. It will be understood that the amount of protein present in the extract is proportional to the total number of mitochondria presented. Thus, the greater the amount of protein the greater the number of mitochondria. In some embodiments, the isolated mitochondria are at a concentration proportional to protein concentration of at least 2, 3, 4, 5, 6, 7, 8, 9, 10, 11, 12, 13, 14, 15, 16, 17, 18, 19, 20, 25, 30, 35, 40, 45, or 50, 75, 80, 90 100, 150, 200, 220, 250, 300, 400, 500, 600, 700, 800, 900 or 1000 ug/ml for every 1 million cells provided. Each possibility represents a separate embodiment of the invention. In some embodiments, the isolated mitochondria are at a concentration of at least 8 ug/ml. In some embodiments, the isolated mitochondria are at a concentration of at least 16 ug/ml.

Methods of determining protein concentration and total protein amounts are well known in the art and any such method may be used. Examples of such methods include, but are not limited to BCA assay, UV-Vis absorbance assay, Western blotting, immunostaining, Bradford assay and protein arrays. In some embodiments, the amount of protein present is determined by Bradford assay.

By another aspect, there is provided a mitochondria extract produced by a method of the invention.

By another aspect, there is provided, a mitochondria extract from a population of hematopoietic cells.

By another aspect, there is provided, a mitochondria extract from a pure population of hematopoietic cells.

By another aspect, there is provided, a mitochondria extract which is pure mitochondria from a single cell type of hematopoietic cells.

In some embodiments, the mitochondria extract is in suspension. In some embodiments, the mitochondria extract is from cells in suspension. In some embodiments, the population of hematopoietic cells is in suspension. In some embodiments, the population is a pure population. In some embodiments, the population is an enriched population. In some embodiments, the population is a mixed population.

In some embodiments, the mitochondria extract is a mitochondria composition. In some embodiments, the extract comprises mitochondria. In some embodiments, the extract comprises isolated mitochondria. In some embodiments, the extract comprises purified mitochondria. In some embodiments, the extract consists essentially of mitochondria. In some embodiments, the extract comprises human mitochondria. In some embodiments, the extract consists of human mitochondria. In some embodiments, the extract comprises mitochondria from a subject. In some embodiments, the extract is hematopoietic cell mitochondria. In some embodiments, the extract is at least 30, 40, 50, 60, 70, 75, 80, 85, 90, 92, 95, 97, 99 or 100% hematopoietic cell mitochondria. Each possibility represents a separate embodiment of the invention. In some embodiments, the extract is immune cell mitochondria. In some embodiments, the extract is at least 30, 40, 50, 60, 70, 75, 80, 85, 90, 92, 95, 97, 99 or 100% immune cell mitochondria. Each possibility represents a separate embodiment of the invention. In some embodiments, the extract is pure T cell mitochondria. In some embodiments, T cell mitochondria are activated T cell mitochondria. In some embodiments, the extract is at least 30, 40, 50, 60, 70, 75, 80, 85, 90, 92, 95, 97, 99 or 100% T cell mitochondria. Each possibility represents a separate embodiment of the invention. In some embodiments, the extract is at least 90% T cell mitochondria. In some embodiments, the extract is at least 95% T cell mitochondria. In some embodiments, the extract is at least 97% T cell mitochondria. In some embodiments, the extract is at least 99% T cell mitochondria. In some embodiments, the extract is pure B cell mitochondria. In some embodiments, the extract is at least 30, 40, 50, 60, 70, 75, 80, 85, 90, 92, 95, 97, 99 or 100% B cell mitochondria. Each possibility represents a separate embodiment of the invention. In some embodiments, the extract is at least 90% B cell mitochondria. In some embodiments, the extract is at least 95% B cell mitochondria. In some embodiments, the extract is pure NK cell mitochondria. In some embodiments, the extract is at least 30, 40, 50, 60, 70, 75, 80, 85, 90, 92, 95, 97, 99 or 100% NK cell mitochondria. Each possibility represents a separate embodiment of the invention. In some embodiments, the extract is at least 90% NK cell mitochondria. In some embodiments, the extract is at least 95% NK cell mitochondria. In some embodiments, the extract is pure HSC mitochondria. In some embodiments, the extract is at least 30, 40, 50, 60, 70, 75, 80, 85, 90, 92, 95, 97, 99 or 100% HSC mitochondria. Each possibility represents a separate embodiment of the invention. In some embodiments, the extract is at least 90% HSC mitochondria. In some embodiments, the extract is at least 95% HSC mitochondria.

In some embodiments, the mitochondria extract comprises a protein concentration of at least 2, 3, 4, 5, 6, 7, 8, 9, 10, 11, 12, 13, 14, 15, 16, 17, 18, 19, 20, 25, 30, 35, 40, 45, 50, 100, 110, 120, 130, 140, 150, 160, 170, 180, 190, 200, 210, 220, 230, 240, 250, 260, 270, 280, 290, 300, 400, 500 or 1000 ug/ml. Each possibility represents a separate embodiment of the invention. In some embodiments, the concentration is for every 1 million cells provided. In some embodiments, the mitochondria extract comprises a protein concentration of at least 8 ug/ml. In some embodiments, the mitochondria extract comprises a protein concentration of at least 16 ug/ml. In some embodiments, the mitochondria extract comprises a protein concentration of at least 100 ug/ml. In some embodiments, the mitochondria extract comprises a protein concentration of at least 110 ug/ml. In some embodiments, the mitochondria extract comprises a protein concentration of at least 200 ug/ml. In some embodiments, the mitochondria extract comprises a protein concentration of at least 220 ug/ml. In some embodiments, the mitochondria extract comprises a protein concentration of about 2, 3, 4, 5, 6, 7, 8, 9, 10, 11, 12, 13, 14, 15, 16, 17, 18, 19, 20, 25, 30, 35, 40, 45, 50, 100, 110, 120, 130, 140, 150, 160, 170, 180, 190, 200, 210, 220, 230, 240, 250, 260, 270, 280, 290, 300, 400, 500 or 1000 ug/ml. Each possibility represents a separate embodiment of the invention. In some embodiments, the mitochondria extract comprises a protein concentration of about 16 ug/ml. In some embodiments, the mitochondria extract comprises a protein concentration of about 110 ug/ml. In some embodiments, the mitochondria extract comprises a protein concentration of about 220 ug/ml.

By another aspect, there is provided a composition comprising a mitochondria extract of the invention.

In some embodiments, the composition is a pharmaceutical composition. In some embodiments, the composition is a therapeutic composition. In some embodiments, the composition consists essentially of the mitochondria extract. In some embodiments, the composition is devoid of an active ingredient or agent other than the mitochondria extract. In some embodiments, the composition consists of the mitochondria extract. In some embodiments, the composition further comprises a pharmaceutically acceptable carrier, excipient or adjuvant. In some embodiments, the composition consists of the mitochondria extract and a pharmaceutically acceptable carrier, excipient or adjuvant. In some embodiments, the composition comprises a therapeutically effective amount of the mitochondria extract. In some embodiments, effective is effective in treating a mitochondria related disease. In some embodiments, the composition is for use in treating a subject. In some embodiments, the composition is for use in treating a condition that benefits from increased mitochondrial function. In some embodiments, the composition is for use in treating a mitochondria related disease. In some embodiments, the composition is for use in treating a disease or condition in a subject in need thereof. In some embodiments, treating a mitochondria related disease comprises producing a medicament for use in treating a mitochondria related disease.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as mannose, lactose, glucose and sucrose, trehalose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The mitochondria of the present invention may be entrapped or encapsulated in a biocompatible natural polymer such as gels or hydrogels, for example hyaluronic acid, chitosan, heparin, alginate, fibrin, collagen, chondroitin sulfate, or silk. The hydrogel can be further formulated with agents for reverse thermal gelation such as Pluronic or methyl cellulose. Synthetic polymers such as poly(ethylene glycol) [PEG], poly(vinyl alcohol) [PVA], poly(N-isopropylacrylamide) [PNIPAAm], and polycaprolactone [PCL] or PCLA-PEG-PCLA can also be used to entrap or formulate the mitochondria for injection in to tissue. In another embodiment the polymer or gel can be a combination of natural and synthetic polymers, in another embodiment the gels or polymers can be crossed linked, or chemically modified to include additional functional groups such as thiols. In some embodiments, the mitochondria are encapsulated or entrapped in a biocompatible polymer. In some embodiments, the polymer is a natural polymer. In some embodiments, the polymer is an artificial polymer.

In another embodiment, the mitochondria of the present invention are encapsulated with particles. In some embodiments, encapsulated with particles is encapsulated in particles. In some embodiments, the particles are lipid particle. In some embodiments, the lipid particle is a liposome. In some embodiments, the lipid particle is a micelle. Any particle delivery method may be used for formulation of the mitochondria for delivery.

In another embodiment, the mitochondria of the present invention may be frozen, or freeze dried.

The carrier may comprise, in total, from about 0.1% to about 99.99999% by weight of the pharmaceutical compositions presented herein.

In some embodiments, the composition is formulated for administration to a subject. In some embodiments, the subject is a mammal. In some embodiments, the mammal is a human. In some embodiments, the subject is a subject in need thereof. In some embodiments, the subject is a subject suffering from a mitochondria related disease. In some embodiments, the subject is a subject suffering from a disease treatable by the mitochondria extract. In some embodiments, the composition is formulated for systemic administration. In some embodiments, the composition is formulated for systemic administration to a subject. In some embodiments, the composition is formulated for local administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include intravascular, intrathecal, intracranial, parenteral, subcutaneous, oral, topical, inhalation, intramuscular, intraocular or intraperitoneal.

The dosage administered will be dependent upon the age, health, and weight of the recipient, kind of concurrent treatment, if any, frequency of treatment, and the nature of the effect desired.

In some embodiments, the composition is formulated for in vitro administration to a cell. In some embodiments, in vitro administration is in vitro transfer to a cell. In some embodiments, to is into. In some embodiments, into a cell is into the cytoplasm of the cell.

By another aspect, there is provided a cell comprising a mitochondria extract of the invention.

In some embodiments, the cell is a recombinant cell. In some embodiments, the cell is not genetically modified. In some embodiments, the cell is of the same species as the mitochondria. In some embodiments, the cell is of the same cell type as the cell type that produced the mitochondria extract. In some embodiments, the cell is of a different cell type than the cell type that produced the mitochondria extract. In some embodiments, the cell is a hematopoietic cell. In some embodiments, the cell is not a hematopoietic cell. In some embodiments, the cell is a cell for adoptive cell transfer (ACT). In some embodiments, the cell is a cell from a subject. In some embodiments, the cell is allogeneic to the subject. In some embodiments, the cell is syngeneic to the subject. In some embodiments, the cell is autologous to the subject.

In some embodiments, the cell is depleted of endogenous mitochondria. In some embodiments, the cell is substantially depleted of endogenous mitochondria. In some embodiments, the cell is devoid of endogenous mitochondria. In some embodiments, substantially depleted comprises less than 50, 45, 40, 35, 30, 25, 20, 15, 10, 5, 4, 3, 2, or 1% of the endogenous mitochondria that had been in the cell. Each possibility represents a separate embodiment of the invention. In some embodiments, substantially depleted comprises less than 20% of the endogenous mitochondria that had been in the cell.

By another aspect, there is composition comprising a cell of the invention.

In some embodiments, the composition further comprises a pharmaceutically acceptable carrier, excipient or adjuvant. In some embodiment, the composition is formulated for administration to a subject. In some embodiments, the composition is formulated for administration to an organ comprising the cells of the same cell type as the cell of the invention. In some embodiments, the composition is for use in ACT.

By another aspect, there is provided a method of treating a subject in need thereof, the method comprising administering to the subject a composition of the invention, thereby treating the subject.

In some embodiments, the subject suffers from a disease or condition. In some embodiments, the disease or condition is an age-related disease or condition. In some embodiments, the disease or condition is a degenerative disease or condition. In some embodiments, the degenerative disease or condition is a neurodegenerative disease or condition. In some embodiments, the disease or condition is a mitochondria related disease or condition. In some embodiments, a mitochondria related disease or condition is a mitochondrial disease or condition. In some embodiments, the disease or condition is treatable by a composition of the invention. In some embodiments, the disease is a metabolic disease or condition. In some embodiments, the disease is an energy homeostasis disease or condition. In some embodiments, the disease is a mitochondrial disease. In some embodiments, the disease is a cardiac disease or condition. In some embodiments, the disease is a cognitive disease. In some embodiments, the disease is a neurodegenerative disease or condition. In some embodiments, the disease is a growth disease or condition. In some embodiments, the disease is a genetic disease or condition. In some embodiments, the disease is a cardiovascular disease or condition. In some embodiments, the disease is a neurophtalmic disease or condition. In some embodiments, the disease is an ophthalmologic disease or condition. In some embodiments, the disease is an ocular disease or condition. In some embodiments, the disease is a corneal disease or condition (e.g., FUCHS Endothelial Corneal Dystrophy). Non-limiting examples of ophthalmologic disease and conditions include Dominant Optic Atrophy (DOA), Leber's Hereditary Optic Neuropathy (LHON), Chronic Progressive External Ophthalmoplegia (CPEO), Neurogenic weakness, Ataxia, Retinitis Pigmentosa (NARP), Mitochondrial encephalomyopathy, Lactic acidosis, and stroke-like episodes (MELAS); Myoclonic epilepsy and Ragged Red Fibers (MERRF), Kearns-Sayre Syndrome (KSS). In some embodiments, the ophthalmic disease is LHON. In some embodiments, the ophthalmic disease is MELAS. In some embodiments the disease is a muscle disease or condition. Non-limiting examples of muscle disorders include MERRF, CPEO, KSS, LHON. In some embodiments the disease is a neurological disease or condition. Non-limiting examples of Neurological disorders include Leigh syndrome, MELAS, KSS, LHON. In some embodiments, the disease is a disease treatable by mitochondrial enrichment therapy. In some embodiments, the disease is a disease treatable by mitochondrial transplantation therapy. In some embodiments, the disease is a disease treatable by mitochondrial replacement therapy. In some embodiments, the disease is a disease treatable by mitochondrial replacement and/or enrichment therapy. In some embodiments, mitochondrial replacement and/or mitochondrial enrichment therapy is mitochondrial donation. In some embodiments, the disease is a systemic disease. In some embodiments, the disease is a multiorgan disease. Non-limiting examples of multiorgan diseases include Leigh syndrome, MELAS, MERRF, KSS, and LHON. In some embodiments, the disease is a multifocal disease. In some embodiments, the disease is an organ specific disease.

In some embodiments, the disease or condition is diabetes. In some embodiments, diabetes is diabetes mellitus. In some embodiments, the diabetes is diabetes type II. In some embodiments, the diabetes is diabetes type I. In some embodiments, the disease or condition is prediabetes. In some embodiments, the disease or condition is elevated blood sugar. In some embodiments, the disease is obesity. In some embodiments, the disease is fatty liver disease. In some embodiments, the fatty liver disease is non-alcoholic fatty liver disease (NAFLD). In some embodiments, the fatty liver disease is steatotic live disease. In some embodiments, NAFLD is metabolic dysfunction-associated steatotic liver disease (MASLD). In some embodiments, the disease or condition is stroke. In some embodiments, the stroke is metabolic stroke. In some embodiments, the disease or condition is seizures. In some embodiments, the disease or condition is cardiomyopathy. In some embodiments, the cardiomyopathy is dilated cardiomyopathy. In some embodiments, the disease or condition is arrhythmia. In some embodiments, the disease or condition is cancer. In some embodiments, the disease or condition is a precancerous malignancy. In some embodiments, the disease or condition is Parkinson's disease. In some embodiments, the disease or condition is Alzheimer's disease. In some embodiments, the disease or condition is amyotrophic lateral sclerosis (ALS). In some embodiments, the disease or condition is multiple sclerosis (MS). In some embodiments, the disease or condition is a genetic mitochondrial disorder. In some embodiments, the genetic disorder is a primary mitochondrial myopathy. In some embodiments, the genetic disorder is mitochondrial encephalopathy. In some embodiments, the genetic disorder is mitochondrial encephalopathy, lactic acidosis and stroke-like episodes (MELAS) syndrome. In some embodiments, the genetic disorder is myoclonic epilepsy with ragged red fibers (MERRF). In some embodiments, the genetic disorder is neuropathy, ataxia and retinis pigmentosa (NARP) syndrome. In some embodiments, the genetic disorder is Leber hereditary optic neuropathy. In some embodiments, the disease or condition is optic neuropathy. In some embodiments, the disease or condition is aging. In some embodiments, the method is a method of treating aging. In some embodiments, the method is a method of improving at least one aging related symptom. In some embodiments, aging comprises muscle aging. In some embodiments, muscle aging comprises muscle atrophy. In some embodiments, the disease or condition is sarcopenia. In some embodiments, aging is skin aging. In some embodiments, aging is ocular aging. In some embodiments, ocular aging comprises macular degeneration. In some embodiments, aging is cognitive aging. In some embodiments, cognitive aging comprises dementia.

In some embodiments, the disease is not a local disease. In some embodiments, the disease is a local disease. In some embodiments, the disease is not ischemia. In some embodiments, the disease is not cardiac ischemia. In some embodiments, the disease is not cardiac disease. In some embodiments, the disease is not cardiac infarction. In some embodiments, cardiac is myocardial. In some embodiments, the disease is not a disease treatable by amounts of mitochondria below 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 mg total. Each possibility represents a separate embodiment of the invention. In some embodiments, the disease is not a disease treatable by amounts of mitochondria below 12 mg total. In some embodiments, the disease is not a disease treatable by amounts of mitochondria below 17 mg total. In some embodiments, the disease is not a disease treatable by amounts of mitochondria below 50 mg total. In some embodiments, the disease is a disease only treatable by amounts of mitochondria above 16, 17, 18, 19, 20, 25, 30, 35, 40, 45, 50, 55, 60, 65, 70, 75, 80, 85, 90, 95 100, 110, 120, 130, 140, 150, 160, 170, 180, 190, 200, 250, 300, 350, 400, 450, 500, 600, 700, 800, 900 or 1000 mg total. Each possibility represents a separate embodiment of the invention. In some embodiments, the disease is a disease only treatable by amounts of mitochondria above 16 mg total. In some embodiments, the disease is a disease only treatable by amounts of mitochondria above 100 mg total. In some embodiments, the disease is a disease only treatable by amounts of mitochondria above 200 mg total. In some embodiments, only treatable is only efficiently treated. In some embodiments, only treatable is clinical benefit is only achievable with the recited amount of mitochondria. In some embodiments, total is the total in a single dose. In some embodiments, total is the total in all doses.

In some embodiments, the extract is autologous to the subject. In some embodiments, the composition is autologous to the subject. In some embodiments, the mitochondria are autologous to the subject. In some embodiments, the extract is allogeneic to the subject. In some embodiments, the composition is allogeneic to the subject. In some embodiments, the mitochondria are allogeneic to the subject. In some embodiments, the extract is syngeneic to the subject. In some embodiments, the composition is syngeneic to the subject. In some embodiments, the mitochondria are syngeneic to the subject.

In some embodiments, the method comprises extracting hematopoietic cells from the subject. In some embodiments, the method comprises taking a blood sample from the subject. In some embodiments, the method comprises receiving a blood sample from the subject. In some embodiments, the method comprises receiving hematopoietic cells from the subject. In some embodiments, the method comprises obtaining a blood sample from the subject. In some embodiments, the method comprises obtaining hematopoietic cells from the subject. In some embodiments, the blood sample is a peripheral blood sample. In some embodiments, the blood sample comprises peripheral blood mononuclear cells (PBMCs). In some embodiments, the hematopoietic cells are PBMCs.

In some embodiments, T cells, B cells, NK cells or HSCs are isolated from the blood sample. In some embodiments, T cells, B cells, NK cells or HSCs are isolated from the hematopoietic cells. In some embodiments, T cells are isolated from the blood sample. In some embodiments, T cells are isolated from the hematopoietic cells. Methods of T cell isolation are well known in the art and any such method or known kit may be used for isolation. For example, isolation may be carried out with an anti-CD3 antibody, anti-CD3 beads or a T cell isolation kit such as are sold by Miltenyi Biotec, Thermo Fisher and many others.

In some embodiments, a mitochondria extract is produced from the blood sample. In some embodiments, a mitochondria extract is produced from the hematopoietic cells. In some embodiments, the mitochondria extract is produced by a method of the invention. In some embodiments, the mitochondria extract is an autologous mitochondria extract. In some embodiments, the mitochondria extract is an allogeneic mitochondria extract. In some embodiments, the mitochondria extract is a syngeneic mitochondria extract.

In some embodiments, the produced mitochondria extract is administered to the subject. In some embodiments, administered is returned. In some embodiments, administered is systemically administered. In some embodiments, administered is by intravenous administration. In some embodiments, administered is local administration. In some embodiments, administered is intravenous administration. In some embodiments, administered is intraocular administration. In some embodiments, administered is administered to a site of disease. It will be understood by a skilled artisan that by using a subject's own blood cells as a source of mitochondria after expansion a cheap, non-immunogenic and inexhaustible source of high-quality mitochondria has been identified. In cases where a subject suffers from a genetic mitochondrial disease or a condition in which all of the subject's mitochondria are effected, it will be necessary to produce the extract from a donors blood cells. However, many diseases characterized by mitochondrial dysfunction feature the disfunction only in a subject set of diseased cells (e.g., liver cells, pancreatic cells) and the mitochondrial in the subject's blood cells would be healthy and usable for therapy.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Isolation of PBMCs from blood samples: Blood samples bags were washed with ethanol, and 13 mL of blood was transferred into 17 mL of RPMI medium and mixed. 13 mL of Ficoll was added to a new tube, and the blood mixture was added to this tube, while holding it at 45° C. The Ficoll-blood tube was centrifuged for 30 min at 800 g at room temperature (RT, 25° C.), with the slowest acceleration and break OFF. The PBMCs layer (the whitish layer formed in the interphase between plasma and Ficoll) was transferred to a new 50 mL tube, followed by filling of the tube to the 45 mL mark with 37° C. RPMI. The PBMCs were centrifuged at 330 g for 10 min at RT, followed by discarding of the supernatant, resuspension of the cells by tapping the tube until no clumps are visible, addition of 5 mL of RPMI, counting of the cells, and resuspension at a concentration of 50×10\hat{ }6 cells/mL in freezing medium (for freezing), or AutoMacs Running Buffer (Miltenyi) for T cell isolation.

Isolation of T cells from PBMCs: Isolation of CD3 positive T cells from PBMCs was done according to the protocol of the Pan T Cell Isolation Kit human (Miltenyi) with several adaptations.

For MACSiBead Particles preparation, 200 μL of CD2-Biotin, 200 μL CD3-Biotin and 200 μL CD28-Biotin were added into a tube and mixed, followed by adding of 1 mL Anti-Biotin MACSiBead Particles and 400 uL of AutoMacs Running Buffer (Miltenyi), and a 2-hour incubation at 4° C. with gentle rotation.

For T cells isolation, PBMCs were suspended in AutoMacs Running Buffer (40 μL of Buffer per 10×10\hat{ }6 total cells), and Pan T Cell Biotin-Antibody Cocktail (10 μL per 10×10\hat{ }6 total cells) was added, followed by a 5-minute refrigerated incubation (2-8° C.). Next, Pan T Cell MicroBead Cocktail (20 μL per 10×10\hat{ }6 total cells) was added and a 10-minute refrigerated incubation was carried out (2-8° C.).

LS Column with a mesh on it was placed in the magnetic field of a suitable MACS Separator, on top of a tube, and was rinsed with 3 mL of Buffer, followed by replacement with a new tube. Cells suspension was applied to the mesh, and the flow-through containing unlabeled cells, representing the enriched T cells was collected. This was followed by washing of the column with 9 mL of Buffer and collecting of the flow-through into the same tube. The column was removed from the separator and placed on a new tube, followed by pipetting of 5 mL buffer, and flushing out the magnetically labeled non-T cells by firmly pushing the plunger into the column.

T cells activation and expansion: Cells were plated in a G-REX 24 well plate or in G-REX 6 well, in suspension cell media (4Cell NutriT media (Gibco)) with or without beads coated with antibodies against human CD2, CD3 and CD28 to mimic antigen-presenting cells and with IL-2 as an activator. Cells were incubated in a 37° C. incubator supplemented with 5% CO2 for 14 days. Medium with IL-2 was renewed at day 4 (6 out of 8 mL) and renewed and completed to 8 mL on days 7, 9, and 11. In the G-REX 6 well, 1-5×10\hat{ }6 cells were seeded in each well and 100 mL of media was added. Cells were incubated in a 37° C. incubator supplemented with 5% CO2. Media was not changed, and IL-2 was added to the well every 3 days. Cell counts were done on day 7, 9, 11 and 14.

MACSiBead Particles (beads coated with antibodies against human CD2, CD3 and CD28, to mimic antigen-presenting cells) were prepared using T Cell Activation/Expansion Kit, human (Miltenyi Biotec, Cat. No. 130-091-441) and MACS Buffer (Miltenyi, Cat. No. 130-091-221). The procedure was done according to the manufacture instructions.

Mitochondria amount: Relative mitochondria amount was determined by testing the mitochondria purified fraction protein concentration with BCA reagent, normalized to the total number of cells used to produce the mitochondria (initial cell plating).

Western blot: Total protein extraction was performed by lysing the cells with RIPA lysis buffer supplemented with protease inhibitor, incubation for 30 min at 4° C., followed by centrifugation at 14,000 g and 4° C. for 10 min, and collection of the supernatant. Isolated mitochondria were used as the mitochondrial sub-fraction. The supernatant above the mitochondrial pellet was used as the cytosolic sub-fraction. Protein concentration was determined by Bradford assay. Samples were separated SDS-PAGE gels, and then the proteins were electro-transferred onto Immobilon-P transfer membrane. The membrane was then blotted with anti-pyruvate dehydrogenase E1α and anti-α-tubulin.

ATP testing: ATP from the expanded T cells was tested with the ATPlite 1-step kit (PerkinElmer), according to the manufacturer's instructions. Briefly, cells were moved to a 96 wells plate in a total volume of 100 uL, and 100 uL of kit reagent was added. Luminescence from whole cell lysate was measured with a plate reader.

Oxygen consumption: Oxygen consumption of the expanded T cells was tested with the MitoXpress Xtra Oxygen Consumption Assay kit (Aligent), according to manufacturer instructions. Briefly, cells were moved to a 96 well plate, followed by addition of kit reagent, sealing of wells with oil and florescence recording (Ex/Em 380/650) with a plate reader. The assay was performed on isolated mitochondria or intact cells.

Membrane potential: Membrane potential was tested using the JC-1 reagent (Sigma), according to manufacturer instructions. Briefly, JC-1 was added to cells (5 uM) for 30 min, followed by 3 washes, and florescence of the cells was recording at Ex/Em 550/600 and 485/535 with a plate reader. The assay was also performed with isolated mitochondria.

Citrate synthase activity: Determining mitochondrial citrate synthase (CS) activity in a sample (either isolated mitochondria or whole cells/cell lysate) was performed via an immunocapture based assay. The assay's principle is capturing the enzyme within the wells of the microplate, and then determining activity by recording color development of TNB, which is generated from DTNB produced in the reaction of citrate synthesis. The overall reaction product, TNB, absorbs at 412 nm. The reaction proceeds as follows:

oxaloacetate+Acetyl CoA+H2O→citrate+CoA-SH+H+

CoA-SH+DTNB→TNB+CoA-S-S-TNB

### (↑ Absorbance at 412 nm)

Antibody ab119692 specifically immunocaptures only native citrate synthase from the applied test sample. In general, this immunocapture based activity assay allows for measuring citrate synthase activity with a simple sample preparation and no need for mitochondria isolation.

Isolation of mitochondria: Cells were centrifuged for 10 minutes at 300×g, washed with PBS, recentrifuged, resuspended with mitochondrial isolation buffer (320 mM sucrose, 5 mM Tris-HCl, pH 7.4, 2 mM EGTA, with or without fatty acid-free BSA (0.5%), protease inhibitor cocktail and PMSF) and homogenized with Dounce homogenizer, with needle shearing (10 passages of the cells through a 30 G needle), or with nitrogen cavitation. Nitrogen cavitation was performed using a disruption vessel (Parr Instruments) with 800 psi for 20 minutes. The cell homogenate was centrifugated at 3000×g for 5 minutes, supernatant was collected and centrifugated again at 3000×g for 5 minutes, and then supernatant was centrifuged at 12,000×g for 10 minutes. The mitochondrial pellet was resuspended in mitochondrial isolation buffer. Mitochondrial concentration was determined by Bradford assay.

Alternatively, mitochondria were isolated using a mitochondria isolation kit (Miltenyi, 130-094-532), according to manufacture instructions. Briefly, cell homogenate was suspended with kit separation buffer, Anti-TOM22 MicroBeads were added, and the suspension was incubated for 1 hour in the refrigerator (2-8°) with gentle shaking. The suspension was then applied onto the kit column, washed with separation buffer, and mitochondria were obtained by removing the column from the separator, adding mitochondrial isolation buffer and pushing the plunger into the column. Mitochondrial concentration was determined by Bradford assay.

Liver analysis: Samples of liver from 36 mice were harvested, fixed in 4% formaldehyde, and kept for 48 hours for further fixation. Then, the tissues were trimmed, put in embedding cassettes, and processed routinely for paraffin embedding. Two cassettes were prepared per animal. Paraffin sections (4 microns thick) were cut, put on glass slides and stained with Hematoxylin & Eosin (H&E) for general histology. In addition, frozen sections (8 microns thick) of liver samples (n=36) were prepared, using a cryostat, and stained with Oil Red-O for detection of triglycerides. The slides were subjected to histopathological evaluation by a pathologist expert in PATHO-LOGICA Ltd.

Pictures were taken, using an Olympus microscope (BX60, serial No. 7D04032) and with the microscope's camera (Olympus DP73, serial No. OH05504) at objective magnification of ×1.25, ×4 and ×10.

The H&E-stained sections were examined, described and scored by the study pathologist, using a semi-quantitative grading scale (5-points scale), for the severity of the histopathological changes (scale is disclosed in Schafer et al., “Use of severity grades to characterize histopathologic changes”, Toxicologic Pathology 2018, 46:256-265, the contents of which are hereby incorporated by reference in their entirety). The scale is as follows:
Grade 0—The tissue appears normal, without any changes at all;
Grade 1—Minimal pathological findings;
Grade 2—Mild pathological findings;
Grade 3—Moderate pathological findings;
Grade 4—Severe pathological findings.

Oli-Red-O Staining for lipid droplets in hepatocytes was scored as follows:
Grade 0=Normal liver with no lipid accumulation;
Grade 1=Very mild accumulation of lipid droplets;
Grade 2=Mild accumulation of lipids;
Grade 3=Moderate accumulation of lipids;
Grade 4=Marked accumulation of lipids.

Statistics: Statistical analysis was conducted using One-Way Analysis Of Variance (ANOVA) followed by Tukey HSD. Significance was considered at p<0.05.

Membrane potential measurement by JC-1: Mitochondrial membrane potential was determined using the cationic dye JC-1 (5,5′,6,6′-tetrachloro-1,1′,3,3′-tetraethylbenzimidazolylcarbocyanine iodide). JC-1 exhibits potential-dependent accumulation in mitochondria, indicated by a shift from green fluorescence (monomers) to red fluorescence (aggregates). Mitochondria were isolated from T cells and resuspended in isolation buffer at a protein concentration of 1 mg/ml, determined by the BCA assay.

Isolated mitochondria were incubated with JC-1 dye at a final concentration of 0.2 μg/ml in assay buffer at 37° C. for 30 minutes to allow for dye uptake and equilibration. Following incubation, fluorescence intensities of JC-1 monomers and aggregates were measured using a fluorescence plate reader with appropriate settings (green fluorescence: Ex/Em=485/535 nm, red fluorescence: Ex/Em=550/600 nm). All assays were performed in triplicate to ensure reproducibility.

### Example 1: Expansion of Primary T Cells for Mitochondria Isolation

In an effort to produce large quantities of functional, therapeutic mitochondria from cells grown in culture it was decided to optimize mitochondrial production/extraction from primary T cells. The viability and integrity of mitochondria is compromised in immortalized cells and mitochondrial damage is a hallmark of cancerous cells, as such primary cells were used even though these cells are in general far more difficult to culture and expand. Hematopoietic cells such as T cells, natural killer (NK cells), B cells and hematopoietic stem cells (HSCs) have the benefit that they grow in suspension and do not adhere to plates. This allows for the culture of a far greater number of cells in the same amount of culture media as would be possible for adherent cells.

T cells were isolated from human PBMCs as described hereinabove (Materials and Methods). Flow cytometry was performed to confirm the homogeneity of the T cell population, and the cultures were seeded with greater than 90% T cells. The isolated T cells were activated with IL-2, then expanded for 2 weeks in a gas permeable rapid expansion (G-REX) plate. These plates are specially designed for suspension cells. The G-REX setup provides unlimited access to nutrients without mixing (decreasing contact with the cells which might result in mitochondrial damage). In particular, there is essentially unlimited access to oxygen as the gas permeable membrane increases oxygen diffusion throughout the media. Flow cytometry is also performed after expansion and the population is found to have greater than 97% purity.

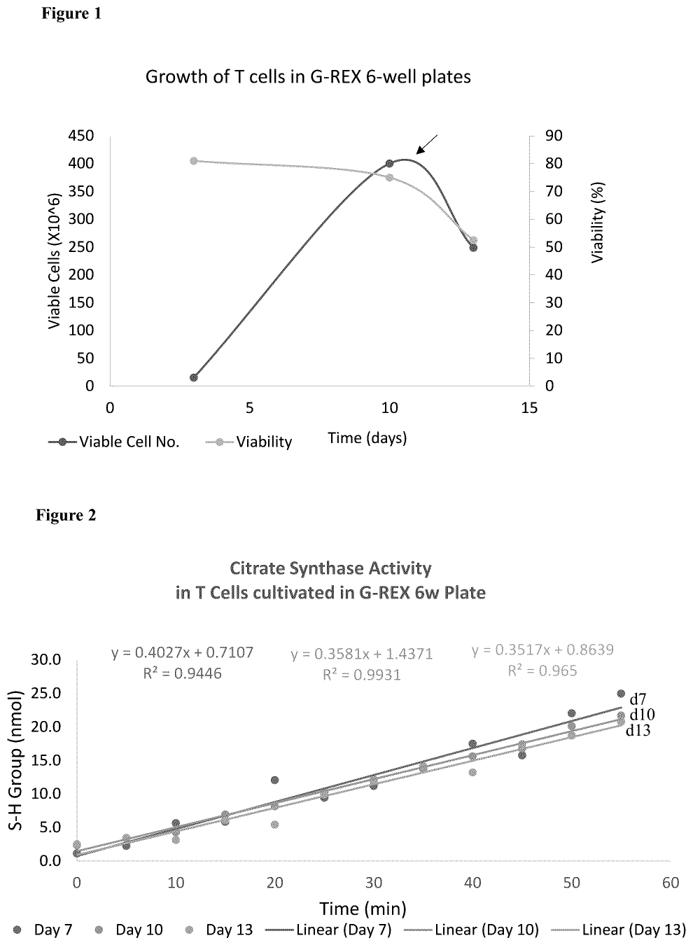

Cell number and viability were monitored across the two weeks (FIG. 1). Cell number was found to peak at 10 days, where 400×10\hat{ }6 cells per 6-well were present. This was an increase of about 80-fold as the initial plating concentration was 5×10\hat{ }6 cells/6-well. Cell viability as assessed by trypan blue staining was also still very high at day 10 but dropped sharply by day 14.

### Example 2: Mitochondria Evaluation and Isolation

Next, mitochondria were isolated from the expanded T cells. It is essential that the isolation process is not overly harsh as this would risk damaging/rupturing the mitochondria. Two methods for cell disruption were tested: Dounce homogenization and nitrogen cavitation. Both methods are known in the art and are generally considered comparable for adherent cells. However, adherent cells must first be trypsinized from the plates and thus are already damaged/ruptured to some extent. Therefore, it was not known if the procedures for adherent cells would be comparable for suspension cells. The results are summarized in Table 1.

TABLE 1
Summary of mitochondrial isolation from expanded T cells

| | | | Mitochondria |
|---|---|---|---|
| | T cells seeded | Yield of | Isolation |
| | in G-Rex 6- | extracted | efficiency |
| Cell disruption | well plate | mitochondria | (ug protein/10\hat{ }6 |
| strategy | (×10\hat{ }6) | (ug protein) | T cells seeded) |
| Nitrogen Cavitation | 5 | 1000 | 220 |
| Dounce homogenizer | 5 | 560 | 112 |

Both methods produced robust yields of intact mitochondria. These yields were far greater than those produced from adherent cells. A ratio of even 220 ug mitochondria from 1 million starting primary suspension cells is unknown in the art, and certainly 90 or 110 ug is well beyond anything that can currently be produced from so few starting cells. Certainly, these amounts of mitochondria cannot be produced when seeding a similar initial amount of primary adherent cells. Culture is also performed in a bioreactor and not in tissue culture cells. Yields from a bioreactor reach and exceed 1000 ug protein from only 10\hat{ }6 starting T cells.

Mitochondria from the expanded T cells were evaluated for quality. This included assaying oxygen consumption, membrane potential, and citrate synthesis (CS) activity. The mitochondria isolated from T cells were found to be functioning and within the expected ranges for all these assays. CS activity was assayed at three time points during the T cell expansion and was found to be comparable at all time points, indicating that prolonged expansion does not negatively impact the mitochondrial quality (FIG. 2 and Table 2).

TABLE 2
Average CS activity over time

| Day of Culture (T Cells) | CS Activity (nmol/min/μL) |
|---|---|
| 7 | 0.032 |
| 10 | 0.029 |
| 13 | 0.028 |

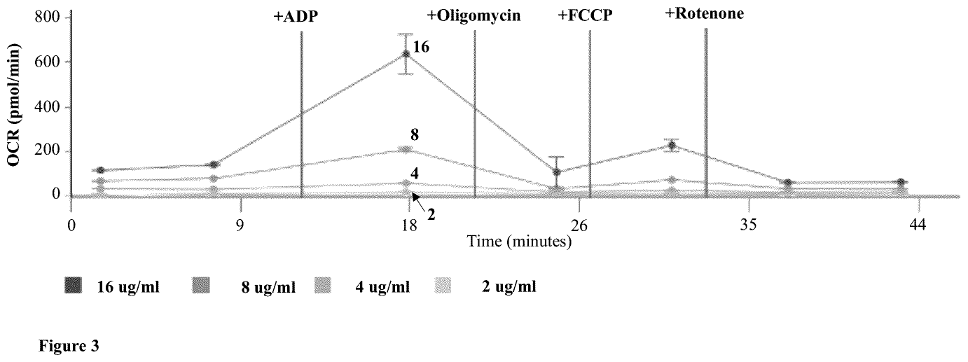

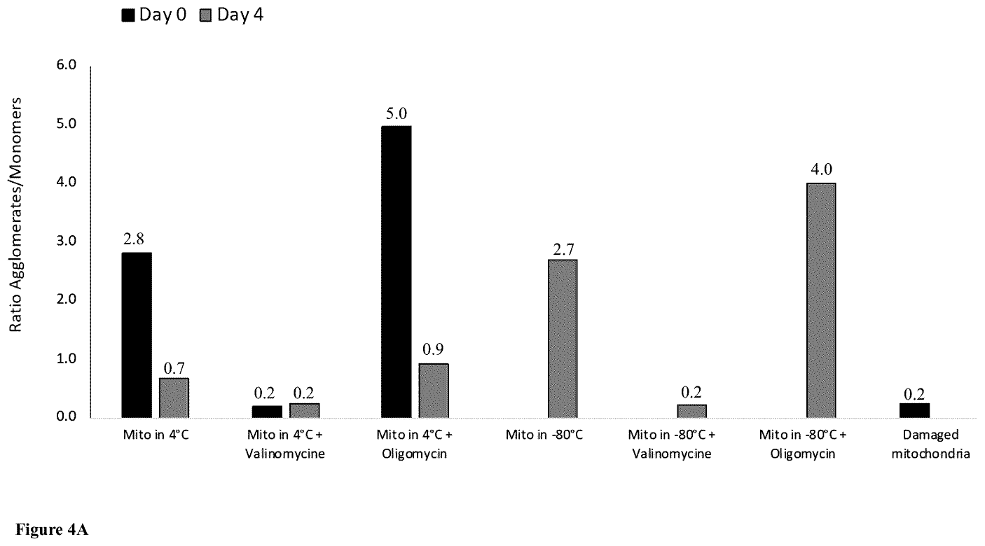

By isolating such a large quantity of mitochondria, it was feasible to produce therapeutic compositions with very high mitochondria concentrations. Mitochondria were isolated from propagating T cells on day 8 from seeding. The oxygen consumption rates (OCR) of isolated mitochondria were tested at four concentrations: 16 ug/mL, 8 ug/mL, 4 ug/mL, and 2 ug/mL in the same total volume. The results are presented in FIG. 3. Throughout the experiment, the substrate glutamate/malate levels were held constant which allows for a direct comparison of mitochondrial activity based on quantity alone. Basal respiration levels were recorded initially. Addition of ADP triggered oxidative phosphorylation, evidenced by an increase in OCR, indicative of ATP production by the mitochondria. This response was concentration-dependent, with the 16 ug/mL preparation displaying the most significant rise, signifying a higher metabolic activity at increased mitochondrial density.

The introduction of oligomycin resulted in a decrease in OCR for all concentrations, consistent with its role as an ATP synthase inhibitor. This demonstrates that the prior increase in OCR was due to ATP synthesis. The uncoupling agent FCCP caused a spike in OCR at all mitochondrial concentrations as expected. This uncoupling agent collapses the proton gradient across the inner mitochondrial membrane, leading to maximal electron transport chain activity without ATP generation. The peak OCR, especially notable in the highest mitochondrial concentration, verifies the capacity for electron transport and suggests robust inner membrane integrity and function. Finally, the addition of rotenone, a complex I inhibitor, brought a decline in OCR in all samples, confirming the role of complex I in the respiratory activity being measured.

### Example 3: Mitochondria Retain their Functionality after Freezing

Membrane potential of isolated mitochondria was tested with JC-1 dye for freshly isolated mitochondria kept at 4° C. directly after isolation, isolated mitochondria kept at 4° C. for 4 days, and isolated mitochondria kept at −80° C. for 4 days. Fresh mitochondria which underwent intentional damaged by several freeze/thaw cycles were used as a negative control.

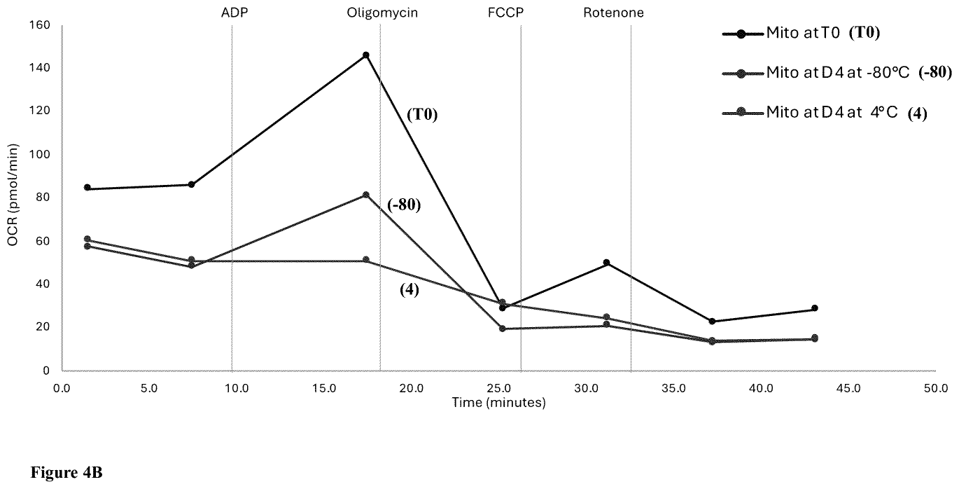

The ratio of red to green fluorescence intensity was calculated for each sample, serving as an indicator of the mitochondrial membrane potential. An increase in the red/green fluorescence ratio indicates a higher membrane potential due to JC-1 aggregation within the mitochondria, whereas a decrease in this ratio suggests mitochondrial depolarization, correlating with a loss of membrane potential. As can be seen in FIG. 4A, membrane potential greatly decreases when mitochondria are stored for 4 days at 4° C., however, a four-day freezing had no appreciable effect on the mitochondria's membrane potential upon thawing. The intentionally damaged mitochondria showed essentially no membrane potential.

For controls, mitochondria were treated, before adding the JC-1 dye, with valinomycin (0.5 μM), a potassium ionophore, to dissipate the membrane potential. This serves as a positive control for depolarization, and as expected, all valinomycin treated mitochondria showed almost no membrane potential. Oligomycin (1 μg/ml), an ATP synthase inhibitor, was used to inhibit the proton flow back into the mitochondria and served as a control to assess the coupling efficiency of the electron transport chain. Blocking ATP synthase leads to an increase in the proton gradient (i.e., an increase in membrane potential). Fresh mitochondria treated with oligomycin indeed showed the expected increase in membrane potential, whereas the mitochondria stored at 4° C. for 4 days were essentially non-responsive to oligomycin, indicating that these mitochondria were not functional. Importantly, the mitochondria stored at −80° C. and then thawed also showed an increase in membrane potential after oligomycin treatment, indicating that −80° C. storage does not significantly impact membrane potential maintenance.

Next, to confirm the functionality of isolated mitochondria that were stored at −80° C. for 4 days, oxygen consumption rate was measured in the Seahorse system. Oxygen consumption rate was significantly reduced when mitochondria were stored at 4° C. (FIG. 4B). Freezing at −80° C. and then thawing, however, only decreased the oxygen consumption rate by a small amount, confirming that the mitochondria is functional and active.

The respiratory control ratio (RCR) is a quantitative measure of mitochondrial efficiency, calculated by dividing the oxygen consumption rate during ADP-stimulated respiration (State 3) by the OCR in the basal, non-phosphorylating condition (State 4). High RCR values indicate efficient coupling of electron transport to ATP synthesis, reflecting healthy mitochondrial function, while low RCR values suggest mitochondrial dysfunction or uncoupling. The RCR of the fresh mitochondria was calculated to be 5, whereas the mitochondria held at 4° C. had an RCR of only 1.6. In contrast, the frozen mitochondria had an RCR of 4.2, indicating that they are still highly functional and active.

### Example 4: Treating Diabetes and NAFLD in a Mouse Model

Type 2 diabetes and non-alcoholic fatty liver disease (NAFLD) are associated with mitochondrial dysfunction and oxidative stress. In diabetic patients, hyperglycemia enhances reactive oxygen species (ROS) production in mitochondria, inducing oxidative damage and impairment of insulin signaling. Mitochondrial biogenesis also modulates energy balance, and excessive ROS generation under high glucose can exacerbate vascular complications. In non-alcoholic fatty liver disease (NAFLD), mitochondrial dysfunction drives abnormal hepatic lipid metabolism and oxidant stress. Lifestyle interventions like diet, exercise and antioxidants provide limited protection as mitochondrial DNA and proteins become irreversibly damaged. Replacement and/or reinforcement of dysfunctional mitochondria with healthy ones could be a promising therapeutic approach for these diseases. As such, the mitochondria composition produced from T cells was tested in a mouse model for its ability to treat both diabetes and NAFLD.

Healthy male C57BL/6J mice, weighing 18-22 g, were used in the study. Animals were maintained under standard housing conditions with access to standard laboratory mouse chow and water. All animal experiments were carried out in accordance with guidelines.

C57BL/6J mice were randomly assigned to six groups (n=6 for each group). Experimental groups are described in Table 3. The mice in groups A and D were fed with a standard chow diet (fat content ˜6%) and served as normal controls, and the mice in the other three groups (B, C, E), received intragastric administration of a high-lard-fat and high-cholesterol diet in which 60% of total calories came from fat.

TABLE 3
Mouse experimental groups

| | | | Diabetes type 2 | |
|---|---|---|---|---|
| | Healthy | | high fat diet | |
| | | # of | | # of |
| Treatment | Group | Animals | Group | Animals |
| Control (saline) | A | 6 | B | 6 |
| Mitochondria treatment, once | | | C | 6 |
| in three days for 3 times | | | | |
| Mitochondria treatment, once | D | 6 | E | 6 |
| every three days for 6 total | | | | |
| administrations | | | | |

8 weeks later, the mice in group D and E were intravenously injected with a formulation of healthy mitochondria isolated from human T cells (0.5 mg/kg body weight, e.g., >9 mg of mitochondrial per injection), once every three days for six total administrations. Mice in group C received only a half treatment with doses administered every three days but for only 3 total treatments. These mice represent the incomplete dosing that is the best possible with more limited sources of mitochondria. For groups A and B, the mice were intravenously administrated an equal volume of saline. Following the final mitochondrial treatment, all mice were fasted for 12 h, and then euthanized by overdose pentobarbital sodium. Mouse serum and liver tissues were collected.

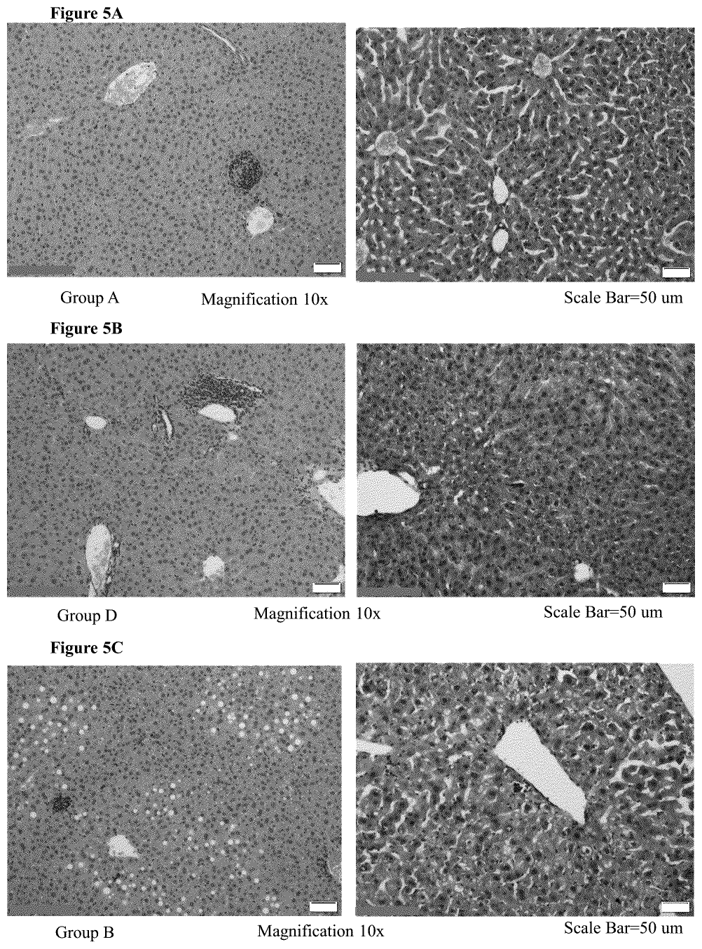

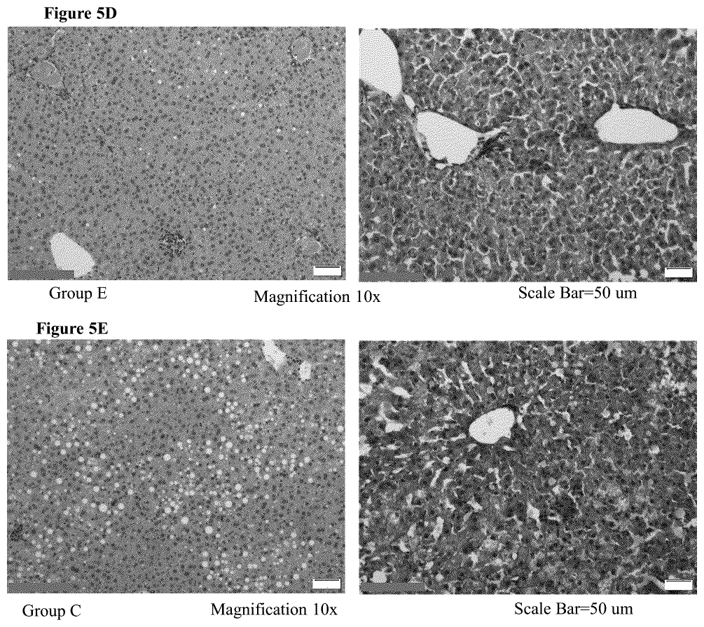

Groups A and D (healthy control animals) displayed normal morphology, without any pathological changes or lipidosis (Scores of 0 for all animals for both pathological severity score and lipidosis) (FIG. 5A-B).

Group B (high fat diet, control) showed a score of 2.16 for both pathological severity score and lipidosis (FIG. 5C). Beside the macro- and micro-vesicular changes in the hepatocytes necrosis of hepatocytes was also observed. H&E staining of the heart was also performed as a control and all animals presented pathology scores of zero. Group E (high fat diet, mitochondria treatment) showed a remarkable improvement with a marked decrease in number and size of the vesicles within the hepatocytes, as well as areas showing regeneration of hepatocytes (FIG. 5D). The Oil Red O staining strongly supported these findings. The grade of the pathological severity (H&E) and the Oil Red O staining was 1.5 and 1.33, respectively. When only a half dosing regime was used (Group C) no improvement was observed (FIG. 5E). Statistical tests showed a significant change between the pathological liver of group B and group E, but group B and group C.

The high total amount of mitochondria used was necessary to produce a systemic effect when administered intravenously. However, as lipid levels and histology were not returned completely to normal, an even higher dosage of mitochondria may be needed. Such a high amount of therapeutic mitochondria was heretofore impossible to isolate. Mitochondrial from mice or other test animals are not suitable for administering therapeutically to humans. Mitochondria from cancerous or immortalized cell lines have damaged mitochondria and also are not suitable for therapeutic administration. Before the method of mitochondria production provided herein, the only suitable methods for producing therapeutic mitochondria were growing adherent primary cells in culture or using a direct tissue biopsy to produce mitochondria. As outlined hereinabove, primary adherent cell culture produced too few cells to feasibly produce such high concentrations and total amounts of mitochondria. Bharadwaj et al., taught the use of percutaneous needle biopsy to produce skeletal muscle tissue for therapeutic mitochondria isolation (see Bharadwaj et al., “Preparation and respirometric assessment of mitochondria isolated from skeletal muscle tissue obtained by percutaneous needle biopsy”, J Vis Exp. 2015; (96): 52350; the contents of which are hereby incorporated by reference in their entirety). As can be seen in FIG. 5 of Bharadwaj the highest total mitochondria yield from one biopsy was about 2 mg. Most produced even less (<1.5 mg).

In the above-described experiments, 0.5 mg/kg mitochondria were administered six times. For an average 70 kg human this is a dose of 35 mg of mitochondria. People suffering from diabetes or NAFLD are likely to be even heavier. A single muscle needle biopsy cannot produce enough mitochondria for even one therapeutic dose let alone 6 (210 mg needed). Further, such treatment might need to be repeated if the subject's diet did not change. It is thus clear that muscle biopsies are not a feasible source of therapeutic mitochondria. Indeed, no source robust enough to produce the therapeutic effects demonstrated herein exists except for the T cell isolation method of the invention.

### Example 5: Comparing Activated T Cells to Mixed PBMCs

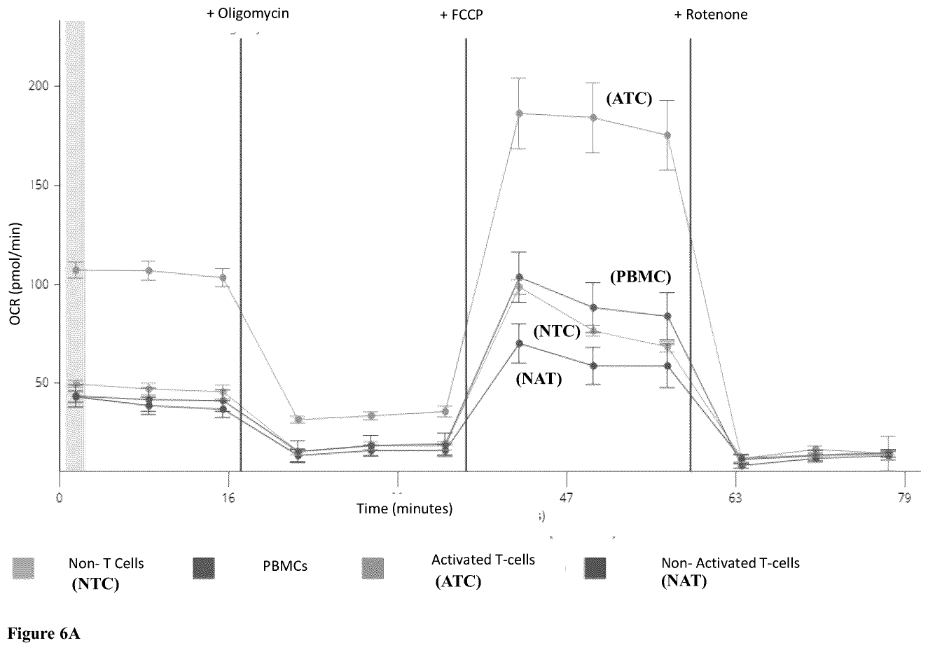

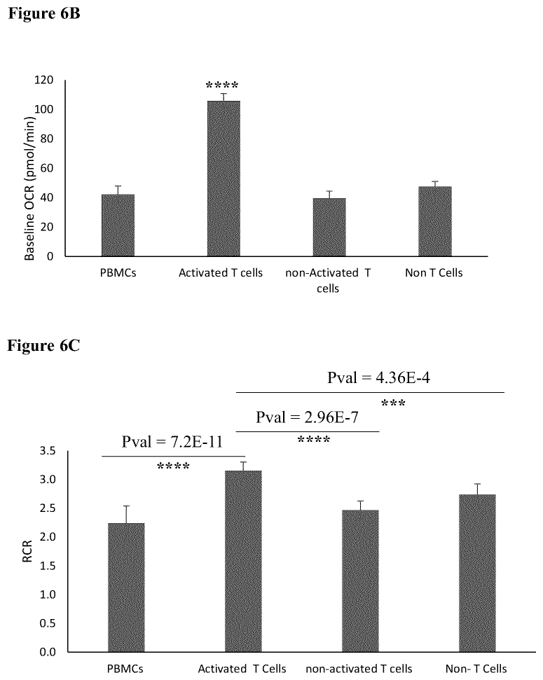

Next, an experiment was performed to compare the quality of mitochondria from activated T cells, non-activated T cells, PBMCs (containing T cells) and PBMCs after T cell isolation (PBMCs without T cells). PBMCs were gathered as described hereinabove. T cells were isolated from the PBMCs as described hereinabove to produce non-activated T cells as well non-T cell PBMCs (referred to hereinafter as non-T cells). Some of the T cells were activated as described hereinabove (CD3/CD28 activation). Seahorse analysis was performed with the different cell types, as done previously to determine the quality and functionality of the mitochondria (FIG. 6A). Surprisingly, the activated T cells had a much higher (and statistically significant) baseline OCR as compared to all the other cells (FIG. 6B). Further, when RCR was calculated the activated T cells had a significantly higher RCR value, indicating superior functionality and quality in the mitochondria in activated T cells (FIG. 6C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for producing a mitochondria extract, the method comprising:
a. providing a population of isolated primary human T cells in suspension;
b. activating said primary human T cells;
c. expanding said activated primary human T cells for a time sufficient to produce an expanded population of human T cells comprising at least 10 times the number of primary human T cells provided; and
d. isolating mitochondria from said expanded population;
thereby producing a mitochondria extract.

2. The method of claim 1, wherein said population of isolated primary human T cells consists of at least 90% T cells.

3. The method of claim 1, wherein said isolated primary T cells are isolated from peripheral blood mononuclear cells (PBMCs).

4. The method of claim 1, wherein said time is between 7 and 14 days.

5. The method of claim 1, wherein said expanded population of human T cells comprises at least 20 times the number of primary human T cells provided.

6. The method of claim 1, wherein said isolated mitochondria comprise at least 20 micrograms of protein for every 1 million primary human T cells provided.

7. The method of claim 1, wherein said activating comprises contacting said primary human T cells with an anti-CD3 antibody.

8. The method of claim 1, wherein said expanding comprises contacting said primary human T cells with at least one of: interleukin-2 (IL-2), an anti-CD3 antibody or antigen-binding fragment thereof, an anti-CD28 antibody or antigen-binding fragment thereof, an anti-CD2 antibody or antigen-binding fragment thereof, and any combination thereof.

9. The method of claim 1, wherein said expanding comprises culturing said suspension of primary human hematopoietic cells in a gas-permeable rapid expansion (G-REX) well.

10. The method of claim 1, wherein said isolating mitochondria comprises lysing cells to produce a cell lysate, wherein said lysing comprises at least one of addition of a lysis buff, needle shearing, homogenization with a Dounce homogenizer and nitrogen cavitation.

11. The method of claim 10, comprising centrifuging said lysate at about 3000 g to remove cellular debris and produce a supernatant and centrifuging said supernatant at about 12,000 g to produce a mitochondrial precipitate.

12. A mitochondria extract produced by a method of claim 1.

13. A mitochondria extract which is at least 90% activated human T cell mitochondria.

14. The mitochondria extract of claim 13, comprising a mitochondrial concentration of at least 16 ug/ml.

15. The method of claim 13, wherein said mitochondria extract comprises at least 90% intact and functional mitochondria capable of oxidative phosphorylation at a rate equal to or greater than the rate of oxidative phosphorylation of mitochondria isolated from control healthy cells.

16. A pharmaceutical composition comprising a mitochondria extract of claim 13 and a pharmaceutically acceptable carrier, excipient or adjuvant.

17. The pharmaceutical composition of claim 16, formulated for systemic administration to a subject.

18. A recombinant cell comprising a mitochondria extract of claim 13.

19. A method of treating a subject suffering from a mitochondrial disease, the method comprising administering to said subject a pharmaceutical composition of claim 16, thereby treating said mitochondrial disease.

20. The method of claim 19, wherein said mitochondrial disease is selected from diabetes, fatty liver disease, non-alcoholic fatty liver disease (NAFLD), Parkinson disease, cancer, Alzheimer's disease, a genetic mitochondrial disorder, aging, and dilated cardiomyopathy.
